SEQUENCE LISTING (1 sequence)

SEQ ID NO: 1 - DNA, 18 bp, Artificial Sequence
  Description of Artificial Sequence Synthetic oligonucleotide
taccgcgtgcgaccctct

(19) United States
(12) Patent Application Publication
(10) Pub. No.: US 2011/0280913 A1
(43) Pub. Date: Nov. 17, 2011

(54) Methods and Compositions for Delivering Therapeutic Agents in the Treatment of B-Cell Related Disorders

(75) Inventors: John C. Byrd, Columbus, OH (US); Natarajan Muthusamy, Galloway, OH (US); Robert J. Lee, Columbus, OH (US); James L. Lee, Columbus, OH (US); Rosa Lapalombella, Dublin, OH (US); Bo Yu, Columbus, OH (US)

(73) Assignee: THE OHIO STATE UNIVERSITY, Columbus, OH (US)

(21) Appl. No.: 13/056,009

(22) PCT Filed: Jul. 28, 2009
(86) PCT No.: PCT/US09/51942

§ 371 Date: Feb. 28, 2011

Related U.S. Application Data
(60) Provisional application No. 61/085,184, filed on Jul. 31, 2008.

(51) Int. Cl. A61K 9/00; C12N 5/0781; A61P 37/04; G01N 33/566; A61K 35/02; A61K 39/395; C12Q 1/02

(52) U.S. Cl. 424/400; 424/133.1; 435/375; 435/29; 435/7.23

(57) ABSTRACT

Methods and compositions for treating and/or diagnosing a B-cell malignancy are described.

### CROSS-REFERENCE TO RELATED APPLICATIONS

This application claims the benefit of U.S. Provisional Application Ser. No. 61/085,184 filed Jul. 31, 2008, the entire disclosure of which is expressly incorporated herein by reference.

### STATEMENT REGARDING FEDERALLY SPONSORED RESEARCH

This invention was made with Government support under Grant Nos. P01 CA95426 and P01 CA101956 from the National Cancer Institute, and EEC-0425626 from the National Science Foundation, and the Government has rights in this invention.

### TECHNICAL FIELD AND INDUSTRIAL APPLICABILITY OF THE INVENTION

The present invention generally relates to compositions and methods for treating and/or diagnosing B-cell malignancies. The present invention also generally relates to methods of treating a B-cell malignancy in a subject, comprising using a sequencing strategy to avoid antagonism between an innate immune enhancing agent comprising lenalidomide and an anti-CD20 antibody comprising rituximab in a therapeutic regimen for the subject. The present invention is also is directed to methods for determining which subjects will most benefit from treatment therapies.

### BACKGROUND OF THE INVENTION

The information provided herein and references cited are provided solely to assist the understanding of the reader and does not constitute an admission that such information or any of the cited references constitute prior art to the present invention.

The anti-CD20 antibody rituximab represents a major therapeutic advance for B-cell malignancies including chronic lymphocytic leukemia (CLL)¹. Rituximab has several potential mechanisms of action including antibody dependent cellular cytotoxicity (ADCC)², complement dependent cytotoxicity (CDC)³, and apoptosis with crosslinking⁴. The importance of ADCC in rituximab efficacy is supported by four non-Hodgkin's lymphoma (NHL) trials in which subjects bearing the FCGR2A-H131R and FCGR3A-V158F high affinity FcγR polymorphisms exhibited improved response to rituximab therapy⁵⁻⁹. While in vitro studies demonstrate rituximab can mediate ADCC against primary CLL cells^{2,10}, one preliminary study did not identify correlation of response with high affinity FcγR polymorphisms¹¹. This has prompted investigation of innate immune enhancing agents to improve both ADCC and rituximab efficacy.

Lenalidomide is one such agent attractive for combination with rituximab. Clinical studies have demonstrated activity in del(5q) myelodysplastic syndrome (MDS)^{12,13}, multiple myeloma¹⁴⁻¹⁶ and CLL^{17,18}. Lenalidomide has been shown to diminish DNA synthesis, promote growth arrest and apoptosis of B-cell lymphoma cell lines without affecting CD20 surface antigen expression^{19,20}. In a Raji cell line xenograft mouse model of disseminated lymphoma, lenalidomide induced NK cell expansion but failed to inhibit tumor growth¹⁹. When lenalidomide and rituximab were combined in this same model, modest prolongation of survival was noted as compared to rituximab monotherapy. Depletion of NK cells in the same model resulted in complete abrogation of in vivo activity, suggesting that murine NK cells are critically important to the mechanism of rituximab and lenalidomide.

Treatment of leukemias such as CLL is very complex. Tremendous clinical variability among remissions is also observed in leukemic subjects, even those that occur after one course of therapy. Subjects who are resistant to therapy have very short survival times, regardless of when the resistance occurs. Despite improvements in outcome with current treatment programs, the need to discover novel agents for the treatment of all types of leukemia continues.

It would be useful to have effective methods of determining whether such treatments were, in fact, efficacious for diseases such as CLL.

A need exists for an effective means to treat CLL and to improve the efficacy of rituximab in those subjects resistant, refractory, or otherwise not responsive to treatment with rituximab.

### SUMMARY OF THE INVENTION

The present invention is also based, in part, upon the discovery that the anti-CD20 antibody rituximab has an antagonistic effect when used with an innate immure enhancing agent such as lenalidomide when used together in the treatment of cancer.

In particular, the inventors herein determined the effects of lenalidomide on CD20 antigen expression as well as determining the effects of rituximab-mediated direct apoptosis and ADCC of CLL cells in vitro.

The inventors' surprising findings, in contrast to the earlier cell line experiments, have important therapeutic implications regarding the therapeutic combination of lenalidomide with rituximab.

Accordingly, the present invention provides a means for treating subjects with a B-cell disorder who need an improvement in the efficacy of, or are not responsive to, a therapeutic regimen that includes the administration of the anti-CD20 antibody rituximab.

The invention provides a method of prevention and/or treatment of a cancer or a tumor, and in particular, to a combination therapy, methods, compositions and pharmaceutical packages comprising an anti-CD antibody and an innate immune enhancing agent.

The present invention provides a method of killing or inhibiting the growth of a tumor cell comprising contacting the cell with an effective amount of said combination of active ingredients.

The present invention also provides a method for treating a subject suffering from a tumor or a cancer, comprising administering to the subject an effective amount of said combination of active ingredients.

The present invention also provides the use of one of the active ingredients in the manufacture of a pharmaceutical for use, in combination with the other active ingredients, in the treatment of a cancer or a tumor.

The present invention also provides a therapeutic combination for the treatment or prevention of a cancer or a tumor.

The present invention also provides a pharmaceutical composition for the treatment or prevention of a cancer or a tumor.

The present invention also provides a packaged pharmaceutical comprising a pharmaceutical composition and instructions to administer an effective amount of one pharmaceutical composition to an individual suffering from a cancer or a tumor, prior to the administration of another, second pharmaceutical composition.

The present invention also provides kits having a combination of active ingredients, with or without pharmaceutically acceptable diluents and carriers, which may be effectively utilized together for carrying out the novel combination therapies of the invention.

The present invention is based, at least in part, on the surprising discovery that rituximab is more effective when not simultaneously administered with the innate immune enhancing agent lenalidomide. Also, a highly synergistic effect can be achieved when such are administered in a temporally sequential manner. This synergistic effect is most pronounced, if the rituximab precedes the lenalidomide agent.

Other systems, methods, features, and advantages of the present invention will be or will become apparent to one with skill in the art upon examination of the following drawings and detailed description. It is intended that all such additional systems, methods, features, and advantages be included within this description, be within the scope of the present invention, and be protected by the accompanying claims.

### BRIEF DESCRIPTION OF THE DRAWINGS

The patent or application file may contain one or more drawings executed in color and/or one or more photographs. Copies of this patent or patent application publication with color drawing(s) and/or photograph(s) will be provided by the Patent Office upon request and payment of the necessary fee.

FIG. 1A: Lenalidomide induces down regulation of CD20 in CLL B cells. CD19+ cells were incubated with Lenalidomide (0.5 μM) or vehicle control. CD20 surface expression was analyzed by flow after 48 h of treatment. The graphs show fold change in % of CD20+ cells (left panel) or MFI (right panel). n=18; * p<0.0001, ** p=0.0015.

FIG. 1B: Panel C shows results for CD20, CD19, CD5 and CD3 surface expression from a representative experiment. Grey line (-) denotes lenalidomide treated cells and dark (-) line denotes vehicle treated cells. Control IgG1k-PE and forward scatter/side scatter plot are shown.

FIG. 1C: Dose and time response curves. The effect of a wider dose range of lenalidomide (0.05-3 μM) on the CD20 and CD52 expression on CLL cells was evaluated at 24 h, 48 h and 72 h.

FIG. 1D: Lenalidomide induces up regulation of CD23 and CD38 in CLL cells. CD19+ cells were incubated with Lenalidomide (0.5 μM) or vehicle control. CD23 and CD38 surface expression was analyzed by flow after 48 h of treatment. The graphs show fold change in % of CD23+ (left) and CD38+ (right) cells. n=6; * p=0.001, ** p<0.05.

FIG. 1E: Lenalidomide induced modulation of CD20 mRNA transcripts in CLL B cells.

FIGS. 2A-2B: Lenalidomide induces CD20 internalization.

FIG. 2A: Cells were incubated with 50 μg/mL of Rituximab to block extracellular CD20, and then washed thrice, fixed and stained with CD20-PE antibody and analyzed by flow. 6 of 8 subjects shown where CD20 antigen down-regulation on the CLL cell surface is accompanied by increased intracellular fluorescence (p=0.006).

FIG. 2B Binding and internalization of anti-CD20 in CLL cells were examined by laser scanning confocal microscopy. CLL cells were incubated with lenalidomide for 48 h at 37° C. Cells were washed, fixed, permeablilized and stained for CD20 (green), concanavalin A (red, DRAQ5 (blue). The images were examined by laser scanning confocal microscopy.

FIG. 2C: Lenalidomide treatment does not alter total level of CD20 protein. CD19+ cells were incubated with Lenalidomide (0.5 μM) or vehicle control. CD20 total protein level was analyzed by western blot after 48 and 72 hours of treatment. Pictures show results for CD20 surface expression from two representative subjects.

FIG. 3A: Lenalidomide enhances intracellular delivery of oligonucleotides in anti-CD20 immunoliposomes. B CLL cells were incubated with rituximab-coated immunoliposomal FAM-ODN for 1 h at 37° C. Cells were washed and incubated with lenalidomide for 1, 4 and 24 h. At the end of the incubation time, cells were washed thrice and visualized on a Nikon fluorescence microscope (magnification, 200×).

FIG. 3B: Lenalidomide enhanced delivery of oligonucleotides requires CD20 antigen binding and internalization. CLL cells were incubated with rituximab, trastuzumab and alemtuzumab coated immunoliposomal FAM-ODN for 1 h at 37° C. Cells were washed and incubated with lenalidomide for an additional 24 h. At the end of the incubation, time cells were washed thrice and visualized on a Nikon fluorescence microscope (magnification, 200×).

FIGS. 4A-4B: Lenalidomide treatment at 48 h significantly decreased rituximab but not alemtuzumab mediated direct cytotoxicity.

FIG. 4A: Direct cytotoxicity: B CLL cells were incubated with 10 gg/mL of rituximab, alemtuzumab or trastuzumab in the presence of 50 μg/mL cross-linking goat anti-human Fc antibody (aFc). Media and crosslinking were used as controls. The percentage of apoptosis was determined by Annexin V/propidium iodide staining after 24 h (N=10, p=0.017). Data shown were normalized on media control.

FIG. 4B: Lenalidomide treatment increases percentage of CD56+/CD1630 cells. Negatively selected NK cells were incubated with Lenalidomide (0.5 μM) or vehicle control. CD56 and CD16 surface expression was analyzed by flow after 72 h of treatment. The graphs show, respectively, percentage change CD56+/CD16+ (left panel, N=12, p<0.01) and CD56−/CD16+ (right panel, N=12, p<0.005) double positive cells.

FIG. 4C: Representative flow results for CD56 and CD16 surface expression of Lenalidomide treated NK cells.

FIGS. 5A-5B: Lenalidomide treatment of allogeneic (a) or autologous (b) derived NK cells results in enhanced rituximab mediated ADCC of untreated CLL cells.

FIG. 5A: ADCC was measured using lenalidomide treated NK cells from normal volunteers and B-CLL cells at 12.5:1, 25:1 and 50:1 effector-to-target ratio (E/T) in the presence or absence of 10 gg/mL rituximab or trastuzumab. Columns, average of triplicate wells and were representative of three independent experiments; bars, SD. The overall Lenalidomide versus vehicle rituximab mediated ADCC was significantly higher for lenalidomide (p=0.009).

FIG. 5B: ADCC was measured using lenalidomide treated NK and B cells from CLL subjects at 12.5:1 and 25:1 effector-to-target ratio (E/T) in the presence or absence of 10 gg/mL rituximab or trastuzumab. Columns, average of triplicate wells and were representative of two independent experiments; bars, SD. (N=2, p=0.02).

FIGS. 6A-6C: Lenalidomide treatment of B-CLL cells resulted in diminished ADCC mediated by rituximab.

FIG. 6A: ADCC was measured using freshly isolated NK cells from normal volunteers and lenalidomide (0.5 μM) or vehicle control treated B-CLL cells at 12.5:1 and 25:1 effector-to-target ratio (E/T) in the presence or absence of 10 μg/mL rituximab or trastuzumab. Columns, average of triplicate wells and were representative of five independent experiments; bars, SD. The overall Lenalidomide versus vehicle rituximab mediated ADCC was significantly lower for lenalidomide (p<0.05).

FIG. 6B: ADCC was measured using untreated NK and lenalidomide or vehicle treated B cells from CLL subjects at 12.5:1 and 25:1 effector-to-target ratio (E/T) in the presence or absence of 10 μg/mL rituximab or trastuzumab. Columns, average of triplicate wells and were representative of two independent experiments; bars, SD. (N=2, p<0.05).

FIG. 6C: ADCC was measured using lenalidomide or vehicle control treated NK and B cells from CLL subjects at 12.5:1 and 25:1 effector-to-target ratio (E/T) in presence or absence of 10 μg/mL rituximab or trastuzumab. Columns, average of triplicate wells and were representative of two independent experiments; bars, SD. (N=2, p=0.02).

FIG. 7: Table 1—clinical characteristics of subjects whose samples were used for in vitro studies.

FIG. 8: Table 2—Fold change in CD20 in B-CLL cells treated with 0/5 μM lenalidomide compared to vehicle. ND: not determined.

### DETAILED DESCRIPTION OF THE PREFERRED EMBODIMENT

Solely for clarity of disclosure, and not by way of limitation, the Detailed Description of the present invention is divided into the following subsections:

I. Definitions

II. Modes for Carrying Out the Invention

III. Pharmaceutical Formulations

IV. Therapeutic Applications

V. Examples

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

### I. Definitions

Throughout this specification, including the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps.

As used herein, “a,” “an” and “the” include singular and plural referents unless the context clearly dictates otherwise. Thus, for example, reference to “an active agent” or “a pharmacologically active agent” includes a single active agent as well as two or more different active agents in combination, reference to “a carrier” includes mixtures of two or more carriers as well as a single carrier, and the like.

Ranges are often expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

A “B cell” is a lymphocyte that matures within the bone marrow, and includes a naive B cell, memory B cell, or effector B cell (plasma cell). The B cell herein is a normal or non-malignant B cell.

A “B-cell malignancy” is a malignancy involving B cells. Examples include Hodgkin's disease, including lymphocyte predominant Hodgkin's disease (LPHD); NHL; follicular center cell (FCC) lymphoma; acute lymphocytic leukemia (ALL); chronic lymphocytic leukemia (CLL); hairy cell leukemia; plasmacytoid lymphocytic lymphoma; mantle cell lymphoma; AIDS or HIV-related lymphoma; multiple myeloma; central nervous system (CNS) lymphoma; post-transplant lymphoproliferative disorder (PTLD); Waldenstrom's macroglobulinemia (lymphoplasmacytic lymphoma); mucosa-associated lymphoid tissue (MALT) lymphoma; and marginal zone lymphoma/leukemia.

The “CD20” antigen, or “CD20,” is an about 35-kDa, non-glycosylated phosphoprotein found on the surface of greater than 90% of B cells from peripheral blood or lymphoid organs. CD20 is present on both normal B cells as well as malignant B cells, but is not expressed on stem cells. Other names for CD20 in the literature include “B-lymphocyte-restricted antigen” and “Bp35.” The CD20 antigen is described in Clark et al., Proc. Natl. Acad. Sci. (USA), 82:1766 (1985), for example. The preferred CD20 antigen is human CD20.

“Treatment” of a subject herein refers to both therapeutic treatment and prophylactic or preventative measures. Those in need of treatment include those already with a B-cell malignancy as well as those in which the B-cell malignancy or the progress of the B-cell malignancy is to be prevented. Hence, the subject may have been diagnosed as having the B-cell malignancy or may be predisposed or susceptible to the B-cell malignancy, or may have B-cell malignancy that is likely to progress in the absence of treatment. Treatment is successful herein if the B-cell malignancy is alleviated or healed, or progression of B-cell malignancy, including its signs and symptoms, is halted or slowed down as compared to the condition of the subject prior to administration.

Successful treatment further includes complete or partial prevention of the B-cell malignancy. For purposes herein, slowing down or reducing B-cell malignancy or the progression of the B-cell malignancy is the same as arrest, decrease, or reversal of the B-cell malignancy. The effectiveness of treatment of the B-cell malignancy in the method can, for example, be determined by using clinical response parameters in the subjects with B-cell malignancies, or by assaying a molecular determinant of the degree of the B-cell malignancy in the subject.

A clinician may use any of several methods known in the art to measure the effectiveness of a particular dosage scheme. Dosage regimens may be adjusted to provide the optimum desired response (e.g., a therapeutic response). For example, a dose may be administered, several divided doses may be administered over time, or the dose may be proportionally reduced or increased as indicated by exigencies of the therapeutic situation.

An “effective response” of a subject or a subject's “responsiveness” to treatment described herein and similar wording refers to the clinical or therapeutic benefit imparted to a subject not responsive to the treatment from or as a result of the treatment. Such benefit includes cellular or biological responses, a complete response, a partial response, a stable disease (without progression or relapse), or a response with a later relapse of the subject from or as a result of the treatment.

The term “further treated”, “further administer” or “further administered”, means that the different therapeutic agents may be administered subsequently, intermittently or one after the other. Such further administration may be temporally separated, for example at different times, on different days, and via different modes or routes of administration.

A “subject” herein is any single human subject, including a subject, eligible for treatment that is experiencing or has experienced one or more signs, symptoms, or other indicators of the B-cell malignancy, whether, for example, newly diagnosed or previously diagnosed and now experiencing a non-response, such as a recurrence or relapse, from a previous treatment. Intended to be included as a subject are any subjects involved in clinical research trials. The subject may be naive to a second medicament being used when the treatment herein is started, i.e., the subject may not have been previously treated with a different agent at “baseline” (i.e., at a set point in time before the administration of a first dose according to the treatment method herein, such as the day of screening the subject before treatment is commenced). Such “naive” subjects are generally considered to be candidates for treatment with such second medicament.

“Clinical improvement” refers to prevention of further progress of the B-cell malignancy or any improvement in the B-cell malignancy as a result of treatment, as determined by various testing.

For purposes herein, a subject is in “remission” if he/she has no symptoms of the B-cell malignancy and has had no progression of the B-cell malignancy as assessed at baseline or at a certain point of time during treatment. Those who are not in remission include, for example, those experiencing a worsening or progression of the B-cell malignancy. Such subjects experiencing a return of symptoms, including active B-cell malignancy, are those who are “non-responsive” or have “relapsed” or had a “recurrence.”

A “medicament” is an active drug to treat the B-cell malignancy or the signs or symptoms or side effects of the B-cell malignancy.

The term “pharmaceutical formulation” refers to a sterile preparation that is in such form as to permit the biological activity of the medicament to be effective, and which contains no additional compositions that are unacceptably toxic to a subject to which the formulation would be administered.

A “package insert” is used to refer to instructions customarily included in commercial packages of therapeutic products or medicaments, that contain information about the indications, usage, dosage, administration, contraindications, other therapeutic products to be combined with the packaged product, and/or warnings concerning the use of such therapeutic products or medicaments, etc.

A “kit” is any manufacture (e.g., a package or container) comprising at least one medicament for treatment of RA or joint damage. The manufacture is preferably promoted, distributed, or sold as a unit for performing the methods of the present invention.

A “target audience” is a group of people or an institution to whom or to which a particular medicament is being promoted or intended to be promoted, as by marketing or advertising, especially for particular uses, treatments, or indications, such as individual subjects, subject populations, readers of newspapers, medical literature, and magazines, television or internet viewers, radio or internet listeners, physicians, drug companies, etc.

### II. Modes for Carrying out the Invention

In one aspect, there is provided herein a method of treating a B-cell malignancy in a subject, comprising using a sequencing strategy to avoid antagonism between an innate immune enhancing agent comprising lenalidomide and an anti-CD20 antibody comprising rituximab in a therapeutic regimen for the subject.

Accordingly, in one embodiment, the present invention relates to a method of treating a B-cell malignancy (e.g., CCL) comprising administering to a subject in need thereof, a first amount of rituximab over a first course of time for a sufficient period of time to have a positive effect, and a second amount of lenalidomide: i) at a second period of time after the first course of time has passed, and ii) for a sufficient period of time to have a positive effect, wherein the first and second amounts together comprise a therapeutically effective amount. For example, in one embodiment, rituximab can be administered to a subject for several days prior to the administration of lenalidomide.

In certain embodiments, the B-cell malignancy comprises chronic lymphocytic leukemia (CLL).

In another aspect, there is provided herein a method for treating a B-cell malignancy in a subject, comprising using lenalidomide therapy to enhance targeted delivery of RNAi-based therapies using a CD20 immunoliposome.

In another aspect, there is provided herein a method of treating a subject in need thereof, comprising using a therapeutic amount of lenalidomide to enhance CD20-liposome-mediated intracellular drug delivery to the subject.

In certain embodiments, the subject is suffering from a B-cell malignancy.

In another aspect, there is provided herein a method of inhibiting a B-cell malignancy cell comprising administering an effective amount of an effective amount of a therapeutic rituximab composition and a lenalidomide therapeutic composition to a cell or subject in need thereof, wherein the administration of lenalidomide does not substantially interfere with the efficacy of the rituximab.

In another aspect, there is provided herein a method of treating a B-cell disorder in a subject in need of such treatment comprising administering to the subject, in therapeutically effective amounts:

a first composition comprising anti-CD20 monoclonal antibody comprising rituximab; and thereafter

a second composition comprising an innate immune enhancing agent comprising lenalidomide that causes one or more of: enhanced CD20 internalization and enhanced NK cell-mediated antibody-dependent cellular cytotoxicity (ADCC), without causing a decrease in efficacy of the first composition.

In certain embodiments, ADCC mediated drug efficacy of rituximab is not substantially diminished.

In another aspect, there is provided herein a method of treating a B-cell malignancy in a subject, comprising:

administering an innate immune enhancing agent comprising lenalidomide in an amount sufficient for causing enhanced CD20 internalization; and

administering one or more CD20 antibody-mediated immune liposomes in an amount sufficient for providing an enhanced ability to deliver oligonucleotide therapy.

In another aspect, there is provided herein a method of treating a B-cell malignancy in a subject, comprising:

sequential administration of an anti-CD antibody comprising rituximab and an innate immune enhancing agent comprising lenalidomide to the subject, wherein the innate immune enhancing agent (lenalidomide) is administered after a period of time sufficient for the anti-CD antibody (rituximab) to induce antibody-dependent cellular cytotoxicity (ADCC); and

subsequent administration of the innate immune enhancing agent (lenalidomide) in an amount sufficient for causing enhanced CD20 internalization and enhanced NK cell-mediated ADCC.

In another aspect, there is provided herein a method of inhibiting or reducing a B-cell malignancy, comprising administering an effective amount of an innate immune enhancing agent comprising lenalidomide sufficient to: i) mediate CD20 internalization and/or ii) enhance intracellular delivery of an oligodeoxynucleotide (ODN) in a rituximab immunoliposome, to a cell, a tissue, or a subject in need thereof.

In another aspect, there is provided herein a method for delivering an ODN to a B-cell comprising using a CD20 immunoliposome composition after the B-cell has been treated with an innate immune enhancing agent comprising lenalidomide.

In another aspect, there is provided herein a method of inhibiting or reducing a B-cell malignancy comprising: screening subjects to identify those in which expression of CD20 is upregulated; and administering an antibody that binds to and inhibits CD20; and thereafter administering an innate immune enhancing agent that internalizes CD20. In certain embodiments, the anti-CD20 antibody comprises rituximab and the innate immune enhancing agent comprises lenalidomide.

In another aspect, there is provided herein a method for increasing sensitivity of a B-cell to a therapeutic agent, comprising contacting the B-cell with a therapeutically effective amount of rituximab, and thereafter contacting the B-cell with a therapeutically effective amount of lenalidomide.

In another aspect, there is provided herein a method for treating a subject for a cancer or pre-malignant condition that is associated with CD20-expressing cells, the method comprising: administering to the subject a therapeutically or prophylactically effective amount of an anti-CD20 antibody comprising rituximab, followed by administering to the subject a therapeutically or prophylactically effective amount of an innate immune enhancing agent comprising lenalidomide. In certain embodiments, the cancer or pre-malignant condition is a cancer of B-cell lineage. In certain embodiments, the cancer of B-cell lineage is chronic lymphocytic leukemia (CLL).

In another aspect, there is provided herein a method for treating a subject for a cancer or pre-malignant condition that is associated with CD20-expressing cells, the method comprising:

administering to the subject a therapeutically or prophylactically effective amount of an anti-CD20 antibody comprising rituximab, such that rituximab is not significantly internalized by CD20-expressing cells following administration, and thereafter

administering an effective amount of an innate immune enhancing agent comprising lenalidomide such that lenalidomide is significantly internalized by CD-expressing cells following administration.

In certain embodiments, the method results in antibody dependent cellular cytotoxicity (ADCC) of CD20-expressing cells.

In another aspect, there is provided herein a method to inhibit one or more B-cell malignancies or metastases in a subject, the method comprising the sequential steps of: a) administering to the subject an effective amount of an anti-CD20 antibody comprising rituximab; b) waiting for an interval of time; and c) administering to the subject an effective amount of innate immune enhancing agent comprising lenalidomide, wherein the B-cell malignancies or metastases decrease more than would be the case for an otherwise identical method that lacks step (c). In certain embodiments, the B-cell malignancies or metastases express a receptor for innate immune enhancing agent. In certain embodiments, step (b) comprises waiting longer than about five days. In certain embodiments, step (b) comprises waiting longer than about ten days.

In certain embodiments, the anti-CD20 antibody and/or the innate immune enhancing agent are administered by introducing into the subject one or more exogenous nucleic acid constructs encoding the anti-CD20 antibody an/or the innate immune enhancing agent, in a manner permitting expression of anti-CD20 antibody and/or innate immune enhancing agent. In certain embodiments, the expression of anti-CD20 antibody and the innate immune enhancing agent are in a time sequenced interval.

In certain embodiments, the interval of time between the administration of the anti-CD20 antibody and the administration of the innate immune enhancing agent is one day to two months.

In another aspect, there is provided herein a method to diagnose the likely responsiveness of a B-cell malignancy to the sequential administration of an anti-CD20 antibody followed by the administration of an innate immune enhancing agent, the method comprising analyzing cells from the B-cell malignancy for the presence of a receptor, wherein the presence of the receptor indicates that the B-cell malignancy will likely respond to the sequential administration of anti-CD20 antibody followed by the administration of the innate immune enhancing agent.

In another aspect, there is provided herein a method for treating a B-cell malignancy by administering to a subject in need thereof an effective amount of an anti-CD20 antibody comprising rituximab and an innate immune enhancing agent comprising lenalidomide in a combined form wherein the rituximab and the lenalidomide are sequentially administered, either close in time or remote in time.

Optionally in such method, at least about one month, preferably at least about two months, and more preferably at least about 52 weeks after the administration, the subject is tested for reduction in the B-cell malignancy.

### III. Pharmaceutical Formulations

Therapeutic formulations used in accordance with the present invention are prepared for storage by mixing the antibody having the desired degree of purity with optional pharmaceutically acceptable carriers, excipients, or stabilizers in the form of lyophilized formulations or aqueous solutions. For general information concerning formulations, see, e.g., Gilman et al., (eds.) (1990), The Pharmacological Bases of Therapeutics, 8th Ed., Pergamon Press; A. Gennaro (ed.), Remington's Pharmaceutical Sciences, 18th Edition, (1990), Mack Publishing Co., Eastori, Pa.; Avis et al., (eds.) (1993) Pharmaceutical Dosage Forms: Parenteral Medications Dekker, New York; Lieberman et al., (eds.) (1990) Pharmaceutical Dosage Forms: Tablets Dekker, New York; and Lieberman et al., (eds.) (1990), Pharmaceutical Dosage Forms: Disperse Systems Dekker, New York, Kenneth A. Walters (ed.) (2002) Dermatological and Transdermal Formulations (Drugs and the Pharmaceutical Sciences), Vol 119, Marcel Dekker.

The preparation of a [first] pharmaceutical composition refers to any process or method performed or required in the generation of a [first] pharmaceutical composition which is ready to be administered to a patient or an individual in need thereof. This includes the manufacture of the pharmaceutical composition, the formulation of the pharmaceutical composition, packaging of the pharmaceutical composition, and other steps performed before the pharmaceutical composition is delivered, requested or made available to a pharmacist, doctor or nurse. It also includes methods and processes performed by the pharmacist, doctor or nurse prior to the administration of the pharmaceutical composition. This includes, for example, dissolving the pharmaceutical composition in an appropriate solvent for administration, e.g. injection, and other steps performed by such a person which aids, facilitates, makes possible or enables the administration of the pharmaceutical composition.

In another aspect, there is provided herein an oral dosage form comprising an effective amount of an anti-CD20 antibody comprising rituximab and an innate immune enhancing agent comprising lenalidomide in a combined form, wherein the oral dosage form is coated with a time (e.g., not pH dependent) release coating material that delays release of the lenalidomide until after rituximab has been released.

In another aspect, there is provided herein an article of manufacture comprising a label and one or more pharmaceutical compositions comprising an effective amount of an anti-CD20 antibody comprising rituximab and an innate immune enhancing agent comprising lenalidomide, wherein the label indicates that the composition(s) is/are for treating a B-cell neoplasm.

Packaging

Another aspect of the invention is to provide a packaged pharmaceutical comprising a pharmaceutical composition and instructions to administer an effective amount of one pharmaceutical composition to an individual suffering from a cancer or a tumor, prior to the administration of another, second pharmaceutical composition.

Another aspect of the invention is to provide kits having a combination of active ingredients, with or without pharmaceutically acceptable diluents and carriers, which may be effectively utilized together for carrying out the novel combination therapies of the invention.

In other embodiments the present invention provides a first pharmaceutical composition as described herein, prepared according to the use described in the preceding paragraphs, included in a pharmaceutical package further including instructions to administer, to an individual suffering from a cancer or a tumor, the first pharmaceutical composition and the second pharmaceutical composition after a prescribed period of time.

In another aspect, there is provided herein a packaged pharmaceutical composition comprising:

a first pharmaceutical composition containing a anti-CD20 antibody comprising rituximab; and

instructions to administer, to a subject suffering from a B-cell malignancy, the first pharmaceutical composition and a second pharmaceutical composition containing an innate immune enhancing agent at an interval of time after the administration of the first pharmaceutical composition.

In another aspect, there is provided herein a packaged pharmaceutical, comprising: a first pharmaceutical composition comprising: (i) an anti-CD20 antibody, or (ii) an innate immune enhancing agent, wherein the packaged pharmaceutical further comprises instructions to administer, to a subject suffering from a B-cell malignancy, the first pharmaceutical composition at a first period of time and a second pharmaceutical composition after an interval of time has passed, the second pharmaceutical composition comprising: in the case of (i), an innate immune enhancing agent, or, in the case of (ii), an anti-CD20 antibody.

In another aspect, there is provided herein a method of treatment of a subject suffering from a B-cell malignancy, the method comprising: administering to the subject a first pharmaceutical composition comprising an (i) anti-CD20 antibody, or (ii) an innate immune enhancing agent, wherein the packaged pharmaceutical composition further comprises instructions to administer, to a subject suffering from a B-cell malignancy, the first pharmaceutical composition at a first period of time and a second pharmaceutical composition after a interval of time has passed, the second pharmaceutical composition comprising: in the case of (i), an innate immune enhancing agent, or, in the case of (ii), an anti-CD20 antibody.

Administration

The composition combination can be formulated and administered to treat individuals with a B-cell malignancy by any means that produces contact of the active ingredients with the agent's site of action in the body of a subject. They can be administered by any conventional means available for use in conjunction with pharmaceuticals, either as individual therapeutic active ingredients or in a combination of therapeutic active ingredients. They can be administered alone, but are generally administered with a pharmaceutical carrier selected on the basis of the chosen route of administration and standard pharmaceutical practice.

In particular embodiments, the administration of the pharmaceutical formulations leads to a situation, in which the rituximab composition is in contact with the rituximab's site of action in the body of a subject, before the lenalidomide composition is in contact with the lenalidomide's site of action in the body of the subject.

In another aspect, there is provided herein a method for the treatment of a subject suffering from a B-cell malignancy, comprising: administering to the subject in need thereof, a first pharmaceutical composition comprising an anti-CD20 antibody, and administering to the subject, a second pharmaceutical composition comprising an innate immune enhancing agent, wherein the second pharmaceutical composition is administered to the subject after an interval of time has passed after the administration of the first pharmaceutical composition.

In another aspect, there is provided herein a method of using a first pharmaceutical composition containing an anti-CD20 antibody agent for the treatment of a subject suffering from a B-cell malignancy, comprising administering an innate immune enhancing agent to the subject after an interval of time has passed after administration of the first pharmaceutical composition containing the anti-CD20 antibody.

In another aspect, there is provided herein a method of using an anti-CD20 antibody for the preparation of a first pharmaceutical composition for the treatment of a subject suffering from a B-cell malignancy, wherein the treatment is a combination treatment comprising: administering the first pharmaceutical composition, and administering a second pharmaceutical composition comprising an innate immune enhancing agent, wherein the second pharmaceutical composition is administered after an interval of time has passed after the administration of the first pharmaceutical composition.

In another aspect, there is provided herein a method of using an innate immune enhancing agent for the preparation of a first pharmaceutical composition for the treatment of a subject suffering from a B-cell malignancy, wherein the treatment is a combination treatment comprising: administering the first pharmaceutical composition, and administering a second pharmaceutical composition comprising an anti-CD20 antibody, wherein the second pharmaceutical composition is administered after an interval of time has passed after the administration of the first pharmaceutical composition.

In another aspect, there is provided herein a kit for administering a first and a second pharmaceutical composition to a subject suffering from a B-cell malignancy, the kit comprising:

a plurality of separate containers, the contents of at least two containers differing from each other in whole or in part, wherein at least one of such containers contains an innate immune enhancing agent, with or without additional pharmaceutical carrier or diluent, and at least one different container contains an anti-CD20 antibody, with or without additional pharmaceutical carrier or diluent; and,

instructions for the use of the contents of the containers after an interval of time has passed after administration of the first pharmaceutical composition for the treatment of a subject suffering from a B-cell malignancy.

In certain embodiments, the container containing the innate immune enhancing agent does not contain an anti-CD20 antibody, and/or wherein the container containing the anti-CD20 antibody does not contain an innate immune enhancing agent.

In certain embodiments, the kit contains instructions where the treatment:

is the sequential administration to the subject of first an anti-CD20 antibody and then an innate immune enhancing agent after an interval of time has passed; or

results in the sequential contact of a B-cell included in, derived from or being part of the B-cell malignancy with first an anti-CD20 antibody and then an innate immune enhancing agent after an interval of time has passed.

In certain embodiments, the kit contains instructions where the treatment:

is the sequential administration to the subject of first an innate immune enhancing agent and then an anti-CD20 antibody after an interval of time has passed; or

results in the sequential contact of a B-cell included in, derived from or being part of the B-cell malignancy with first an innate immune enhancing agent and then an anti-CD20 antibody after an interval of time has passed.

In certain embodiments, the administration: is the sequential administration to the subject of an anti-CD20 antibody and an innate immune enhancing agent within from 1 day to 10 days of each other; or results in the sequential contact of a cell included in, derived from, or being part of, the B-cell malignancy with an anti-CD20 antibody and an innate immune enhancing agent within from 1 day to 10 days of each other.

In certain embodiments, the administration: is the sequential administration to the subject of the anti-CD20 antibody and the innate immune enhancing agent after an interval of time has passed; or results in the sequential contact of a B-cell included in, derived from or being part of the B-cell malignancy with the anti-CD20 antibody and the innate immune enhancing agent after an interval of time has passed.

In certain embodiments, the administration: is the sequential administration to the subject of first the innate immune enhancing agent and then the anti-CD20 antibody after an interval of time has passed; or results in the sequential contact of a B-cell included in, derived from or being part of the B-cell malignancy with first the innate immune enhancing agent and then the anti-CD 20 antibody after an interval of time has passed.

In certain embodiments, the interval of time between the administration of the anti-CD20 antibody and the administration of the innate immune enhancing agent is one day to two months.

In certain embodiments, the administration: is the sequential administration to the subject of the anti-CD20 antibody and the innate immune enhancing agent within about 10 days, 7 days, 5 days, 3 days, 2 days or 1 day of each other; or results in the sequential contact of a B-cell included in, derived from or being part of the B-cell malignancy with the anti-CD20 antibody and the innate immune enhancing agent within about 10 days, 7 days, 5 days, 3 days, 2 days or 1 day of each other.

In certain embodiments, the administration: is the sequential administration to the subject of the anti-CD20 antibody and the innate immune enhancing agent within about 48 hours, 24 hours, 12 hours, 8 hours, 6 hours, 4 hours, 2 hours, 1 hour, 30 mins, 15 mins or 5 mins of each other; or results in the sequential contact of a B-cell included in, derived from or being part of the B-cell malignancy with the anti-CD20 antibody and the innate immune enhancing agent within about 48 hours, 24 hours, 12 hours, 8 hours, 6 hours, 4 hours, 2 hours, 1 hour, 30 mins, 15 mins or 5 mins of each other.

### IV. Therapeutic Applications

The composition(s) described herein may be administered in any dose, provided it is effective to treat the subject. A physician having ordinary skill in the art can readily determine and prescribe the effective amount of the pharmaceutical composition(s) required, depending on such factors as the particular composition(s) employed, prior clinical experience published in the literature on the composition(s) employed, the subject's characteristics and clinical history, the type and severity of the disorder or disease, other medicines being given, and any side effects predicted. For example, the physician could start with doses of a composition(s), employed in the pharmaceutical composition at levels lower than that required in order to achieve the desired therapeutic effect and gradually increase the dosage until the desired effect is achieved. The effectiveness of a given dose or treatment regimen of the composition(s) can be determined, for example, by assessing signs and symptoms and/or assessing inhibition of structural damage or of radiographic progression in the subject using the standard measures of efficacy.

The dose may be by weight or a fixed dose, preferably a fixed dose regardless of weight. As a general proposition, the effective amount of the composition(s) administered parenterally per dose will be in the range of about 20 mg to about 5000 mg, by one or more dosages, which can be translated to a dose by weight. These doses may be given as a single dose or as multiple doses, for example, two to four doses. Such doses may be done by infusions, for example. However, these suggested amounts of the composition(s) are subject to a great deal of therapeutic discretion. The key factor in selecting an appropriate dose and scheduling is the result obtained, as indicated above. For example, relatively higher doses may be needed initially for the treatment of ongoing and acute disorder or disease. To obtain the most efficacious results, the composition(s) are administered as close to the first sign, diagnosis, appearance, or occurrence of the disorder or disease as possible or during remissions of the disorder or disease.

In one preferred embodiment, one may administer a second medicament, as a consecutive administration in either order, wherein preferably there is a time period while both (or all) active agents to sequentially exert their biological activities without reducing the efficacy of the other composition.

Many alternative experimental methods known in the art may be successfully substituted for those specifically described herein in the practice of this invention, as, for example, described in manuals, textbooks and websites available in the areas of technology relevant to this invention (e.g., Using Antibodies, A Laboratory Manual, Harlow, E. and Lane, D., eds. (Cold Spring Harbor Laboratory Press, New York, 1999); Roe et. al., DNA Isolation and Sequencing (Essential Techniques Series) (John Wiley & Sons, 1996); Methods in Enzymology: Chimeric Genes and Proteins, Abelson et al., eds. (Academic Press, 2000); Molecular Cloning: a Laboratory Manual, 3rd Edition, by Sambrook and MacCallum, (Cold Spring Harbor Laboratory Press, New York, 2001); Current Protocols in Molecular Biology, Ausubel et. al., eds. (John Wiley & Sons, 1987) and periodic updates; PCR: The Polymerase Chain Reaction, (Mullis et al., ed., 1994); Current Protocols in Protein Science, Coligan, ed. (John Wiley & Sons, 2003); and Methods in Enzymology: Guide to Protein Purification, Vol. 182, Deutscher, ed. (Academic Press, Inc., 1990))

In a particular aspect, the present invention relates to methods for the treatment of cancer. In one embodiment, the cancer to be treated is chronic lymphocytic leukemia (CLL).

In the context of the methods of treatment described herein which refer to two active agents (e.g., the anti-CD2-antibody (i.e., rituximab) and the innate immune enhancing agent (i.e., lenalidomide), as described herein), the term “therapeutically effective amount” is intended to qualify the combined amount of the first and second agents in the “sequential administration” therapeutic regimen.

The combined, sequentially administered amount will achieve the desired biological response, for example, partial or total inhibition, delay or prevention of the progression of cancer including cancer metastasis; inhibition, delay or prevention of the recurrence of cancer including cancer metastasis; or the prevention of the onset or development of cancer (chemoprevention) in a subject, for example a human.

In the context of the methods of treatment described herein which refer to a single active agent, the term “therapeutically effective amount” is intended to qualify the amount of that agent used in therapy. The amount will achieve the desired biological response, for example, partial or total inhibition, delay or prevention of the progression of cancer including cancer metastasis; inhibition, delay or prevention of the recurrence of cancer including cancer metastasis; or the prevention of the onset or development of cancer (chemoprevention) in a subject, for example a human.

In some embodiments, the “sequential administration” combination therapy results in a synergistic effect, for example, the (e.g., the anti-CD2-antibody (i.e., rituximab) and the innate immune enhancing agent (i.e., lenalidomide), act synergistically when administered in a sequential manner, for example, in the inhibition of the proliferation of cancer cells, for example, multiple myeloma tumor cells.

For example, in one embodiment, the first treatment procedure can take place prior to the second treatment procedure (i.e., administration of the other chemotherapeutic agent), after the second treatment procedure. In one example, the method includes a first step of administering a therapeutic amount of rituximab, and a second step of administering a therapeutic amount of lenalidomide can take place sequentially in any order, as long as one agent does not interfere with the efficacy of the other agent.

For example, a total treatment period can be decided for the rituximab. The lenalidomide can be administered following treatment with the rituximab. In addition, other chemotherapeutic agents can be administered, but do not need to occur over the entire treatment period.

One surprising and unexpected result disclosed herein is the ability of active agents (e.g., the anti-CD2-antibody (i.e., rituximab)) and the innate immune enhancing agent (i.e., lenalidomide), to act synergistically in the inhibition of the proliferation of ovarian tumor cells when administered in a sequential, temporally spaced manner.

Also, it is understood that the sequential administration combination therapies described herein can provide a therapeutic advantage in view of the differential toxicity associated with the individual treatment modalities. As such, this differential toxicity can permit each treatment to be preferably administered at a lower therapeutic dose, without increasing subject morbidity.

Inhibiting Cell Proliferation, Etc.

As discussed herein, one aspect of the present invention is a method (e.g., an in vitro method, an in vivo method) for selectively inducing ADCC, CDC, and/or apoptosis of, or reducing the viability of, a neoplastic cell characteristic of B-cell malignancy (e.g., leukemia cell), wherein the method comprises contacting the cell with an effective amount of the active agents (e.g., the anti-CD2-antibody (i.e., rituximab)) and the innate immune enhancing agent (i.e., lenalidomide).

Another aspect of the present invention is a method (e.g., an in vitro method, an in vivo method) of preventing, inhibiting (fully or partially), or arresting cell proliferation of a neoplastic cell characteristic of a B-cell malignancy (e.g., a leukemia cell), wherein the method comprises contacting the neoplastic cell with an effective amount of the an active agent (e.g., the anti-CD2-antibody (i.e., rituximab)) and the innate immune enhancing agent (i.e., lenalidomide).

In one embodiment, the method is practiced in vitro. In another embodiment, the method is practiced in vivo. In one embodiment, the neoplastic cell is a leukemia cell. In one embodiment, the neoplastic cell is in (or is part of) a subject (e.g., a living subject) such as a subject, for example, a human.

The value of the present invention can thus be seen by reference to the Examples herein.

### V. Examples

The present invention is further defined in the following Examples, in which all parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. All publications, including patents and non-patent literature, referred to in this specification are expressly incorporated by reference. The following examples are intended to illustrate certain preferred embodiments of the invention and should not be interpreted to limit the scope of the invention as defined in the claims, unless so specified.

Cell Isolation

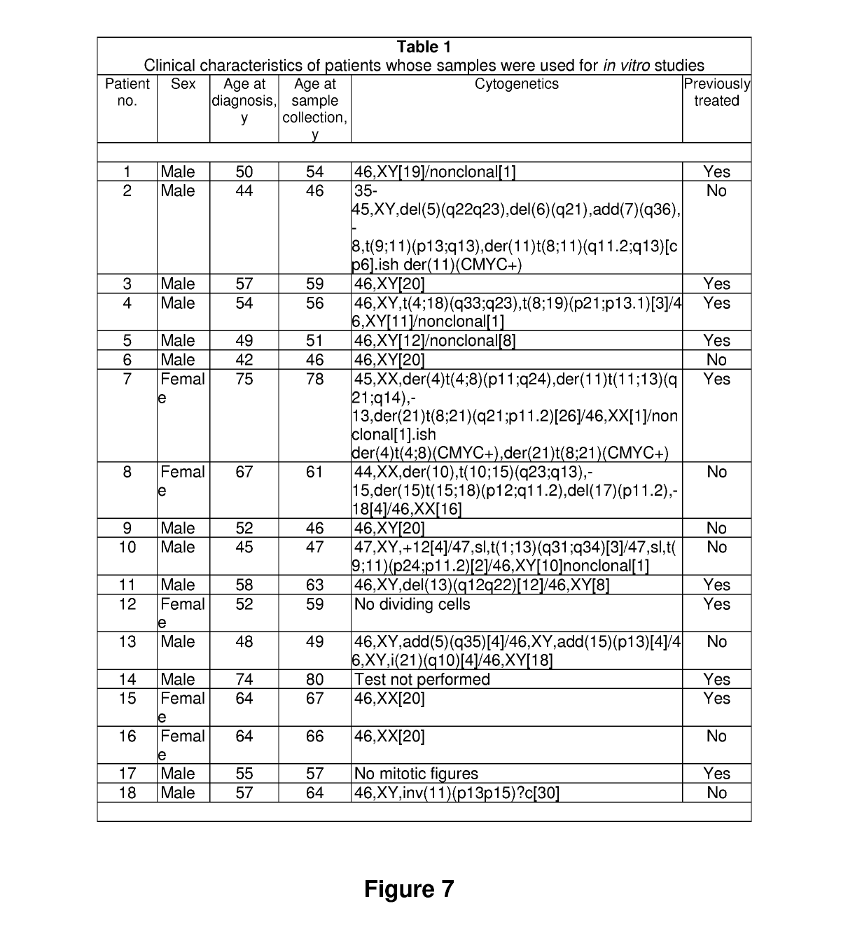

Blood was obtained from subjects with CLL as described by National Cancer Institute (NCI) Working Group criteria.²¹ All subjects provided informed consent under an Ohio State University Institutional Review Board-approved protocol in accordance with the Declaration of Helsinki. The clinical features of each of these subjects are summarized in FIG. 7—Table 1.

CLL B cells were isolated by Ficoll centrifugation using Rosette-Sep reagent (StemCell Technologies, Vancouver, BC) according to the manufacturer's instructions. CLL cells were incubated in RPMI 1640 medium supplemented with 10% heat-inactivated human serum (Valley Biomedical, Winchester, Va.), 2 mM L-glutamine (Invitrogen, Carlsbad, Calif.), and 100 U/mL penicillin/100 μg/mL streptomycin (Sigma-Aldrich, St Louis, Mo.) at 37° C. in 5% CO₂.

For ADCC experiments, CD56⁺ and CD19⁺ cells were negatively purified from whole blood obtained from either healthy volunteers or CLL subjects by magnetic-activated cell sorting according to the manufacturer's recommendations (MiniMACS; Miltenyi Biotec, Auburn, Calif.). The purity of enriched populations was always greater than 95% of the total yield as detected by CD19 and CD3 staining.

Lenalidomide Extraction and Purification

The lenalidomide used for the in vitro CLL experiments was extracted from commercial capsules donated by several subjects who had stopped treatment. Powdered material was stirred in a mixture of 250 mL ethyl acetate with 10 mL triethylamine for 3 hours and then filtered. This process was repeated with the residual powder 2 additional times. The collected organic solvent was dried to yield a white solid, which was used for biochemical testing. The purity of capsule-extracted lenalidomide was evaluated with nuclear magnetic resonance (NMR) and liquid chromatography/mass spectrometry in The Ohio State University Pharmacoanalytical Shared Resource as published.²² The NMR spectra contained only lenalidomide resonance peaks and had no indication of contaminating materials. Powdered lenalidomide was then resuspended in phosphate-buffered saline (PBS), pH 1.4 (vehicle). The same buffer was used as control in all the in vitro studies. When diluted to the equivalent of 0.5 μM in the media, the pH is 7.4.

Immunophenotyping Studies

Immunophenotyping was performed to determine the percentage and mean fluorescence intensity of CD20, CD19, CD5, CD23, CD38, CD3, and CD52 on CLL cells relative to a specific isotype control. Cells (10⁶) were washed in PBS, stained with fluorochrome-labeled antibodies at 4° C., then rinsed in PBS and analyzed by flow cytometry (EPICS-XL; Beckman Coulter, Fullerton, Calif.). A total of 10,000 events were collected and analyzed using Windows Multiple Document Interface for Flow Cytometry (WinMDI). CD20, CD19, CD3, CD5, CD23, and CD38 antibodies were obtained from BD Biosciences (San Jose, Calif.), and CD52 antibody was obtained from Serotec (Raleigh, N.C.).

Intracellular CD20 Staining

CLL cells were incubated with a molar excess of rituximab to saturate the binding of extracellular CD20. Cells were washed twice with PBS and then fixed and permeablilized using the Cytofix/Cytoperm Kit (BD Biosciences PharMingen, San Diego, Calif.) according to the manufacturer's recommendations. Cells were then incubated with CD20-phycoerythrin (PE) antibody, washed twice in PBS, and analyzed by flow cytometry. To ensure the specificity of the staining procedure, binding of IgG1-k isotype antibody was also measured.

Immunoblotting

Immunoblot assays were performed with the multiple antigen detection immunoblotting method as described previously.²³ Whole-cell lysates were prepared and stored in −80° C. Protein concentration was quantified by the bicinchoninic acid method (Pierce Chemical, Rockford, Ill.). Protein samples (50 μg) were separated by sodium dodecyl sulfate-polyacrylamide gel electrophoresis (12%) and transferred to 0.2-μm nitrocellulose membranes (Whatman Schleicher & Schuell, Keene, N.H.) and incubated with goat antihuman CD20 (Santa Cruz Biotechnology, Santa Cruz, Calif.) or rabbit antiactin (Cell Signaling Technology, Danvers, Mass.) antibodies. Horseradish peroxidase-conjugated goat anti-rabbit IgG (Bio-Rad, Hercules, Calif.) was used as secondary antibody Immune complexes were detected using chemiluminescent substrate (SuperSignal; Pierce Chemical).

Analysis of Cytotoxicity in Presence of Cross-linking

Cells (10⁶ cells/mL) were treated with commercially purchased alemtuzumab (anti-CD52; Ilex Pharmaceuticals, San Antonio, Tex.), rituximab (anti-CD20; Genentech, San Francisco, Calif.), or trastuzumab (anti-HER2; Genentech) at a concentration of 10 μg/mL. Goat antihuman IgG cross-linker (Fc specific; Jackson ImmunoResearch Laboratories, West Grove, Pa.) was added to the cell suspension 5 minutes after adding the primary antibodies at a concentration of 50 μg/mL.

In addition, samples with no treatment or crosslinker alone were collected at matched time points. Apoptosis of cells at 24 and 48 hours after treatment was measured by annexin V-fluorescein isothiocyanate/propidium iodide (PI) flow cytometry analysis according to the supplier's instructions (BD Biosciences).

Cytotoxicity results were expressed as percentage of total positive cells over untreated control [% positive cells=(% annexin⁺ and/or PI⁺ cells of treatment group)−(% annexin⁺ and/or PI⁺ cells of untreated control)].

Analysis of ADCC

ADCC activity was determined by standard 4-hour ⁵¹Cr-release assay. ⁵¹Cr-labeled target cells (10⁵ CLL cells/mL) were incubated with media alone or in the presence of various antibodies (10 μg/mL) at 37° C. for 30 minutes. Unbound antibody was washed off and the cells plated at 10⁴ cells/well. Effector cells (natural killer [NK] cells from healthy donors or CLL subjects) were then added to the plates at indicated effector-to-target (E:T) ratios. After a 4-hour incubation, supernatants were removed and measured using a gamma counter. The percentage of specific cell lysis was determined by the equation: % lysis=100×(ER−SR)/(MR−SR), where ER, SR, and MR represent experimental, spontaneous, and maximum release, respectively. Data were normalized to the untreated control.

Semiquantitative Reverse-transcribed Polymerase Chain Reaction

RNA was extracted with TRIzol reagent (Invitrogen) according to the manufacturer's directions and converted to cDNA with the SuperScript First-Strand Synthesis System (Invitrogen). Real-time polymerase chain reaction was performed using custom-designed primers for CD20 (ID: Hs00544819_m1; Applied Biosystems, Foster City, Calif.) and an ABI Prism 7700 sequence detection system (Applied Biosystems). The expression of CD20 relative to an internal control gene was calculated by plotting the Cₜ (cycle number), and the average relative expression for each group was determined using the comparative method (2^{-Ct}).²⁴

Preparation of Antibody-coated Immunoliposomes

3β-[N-(N′,N′-Dimethylaminoethane)-carbamoyl] cholesterol (DC-Chol) and DSPE-PEG-maleimide (DSPE-PEG-Mal) were purchased from Avanti Polar Lipids (Alabaster, Ala.). Methoxy-polyethylene glycol (molecular weight=2000 Da)-distearoyl phosphatidylethanolamine (DSPE-PEG) and egg phosphatidylcholine (Egg PC) were obtained from Lipoid (Newark, N.J.). 2-Iminothiolane (Traut reagent), 5,5′-dithiobis-(2-nitrobenzoic acid) (Ellman reagent), and other chemicals were purchased from Sigma-Aldrich. A carboxyfluorescein (FAM) terminus modified oligodeoxynucleotide (ODN) (5′-(6) FAM-TAC CGC GTG CGA CCC TCT) [SEQ ID NO:1] was custom-synthesized by Alpha DNA (Montreal, QC).

An ethanol dilution method was modified to prepare the FAM-ODN-loaded immunoliposomes.²⁵ Briefly, protamine sulfate in citric acid (20 mM, pH 4) was mixed with lipids (DC-Chol/Egg-PC/PEG-DSPE [molar ratio]=28.0:70.0:2.0) at a mass ratio of lipids:protamine=12.5:0.3, followed by addition of ODN in citric acid (20 mM, pH 4) at ODN/lipids/protamine (weight ratio)=1:12.5:0.3. The complexes were then dialyzed against citric acid (20 mM, pH 4) for 1 hour and then further dialyzed against N-2-hydroxyethylpiperazine-N′-2-ethanesulfonic acid-buffered saline (145 mM NaCl, 20 mM N-2-hydroxyethylpiperazine-N′-2-ethanesulfonic acid, pH 7.4) overnight, using a DispoDialyzer (Spectrum Labs, Rancho Dominguez, Calif.) with a molecular weight cutoff of 10,000 Da. Liposome size distribution was analyzed by dynamic light scattering on a NICOMP Particle Sizer Model 370 (Particle Sizing Systems, Santa Barbara, Calif.). Volume-weighted analysis showed an average particle size of approximately 50 nm.

A postinsertion method was adopted to incorporate antibody ligands into preformed liposomes containing FAM-ODN. In this method, rituximab, trastuzumab (anti-Her2), or alemtuzumab (anti-CD52) was incubated with 20× Traut reagent (2 hours at room temperature) to yield sulfhydryl-modified antibodies. The anti-CD20—sulfhydryl was then incubated with micelles of Mal-PEG-DSPE at a molar ratio of 1:10 and then incubated with FAM-ODN loaded liposomes for 1 hour at 37° C. Targeted liposomes with antibody-PEG-DSPE-to-lipid ratios of 1:1000 (0.1 mol/dL) were thus prepared.

Rituximab Immunoliposome Uptake Studies

CLL cells were incubated at 10⁶ cells/mL for 1 hour at 37° C. with rituximab-, trastuzumab-, or alemtuzumab-coated immunoliposome-FAM-ODN (C_{ODN}=1.0 μM) and washed twice in PBS. Cells were kept in culture in RPMI 10% fetal bovine serum for an additional 1, 4, and 24 hours, respectively, in the presence of either 0.5 μM lenalidomide or vehicle control. Cells were then washed once with cold 0.5 M glycine buffer (pH 2.8) containing 0.15 M NaCl followed by cold PBS wash to remove extracellular immunoliposome particles resulting from acid wash has been shown to induce detachment of antibody from antigen.^{26,27} This was further verified by flow cytometry (data not shown). Cells were visualized on a Nikon fluorescence microscope (magnification ×200).

Laser-scanning Confocal Microscopy

Binding and internalization of anti-CD20 in CLL cells were examined by laser scanning confocal microscopy. CLL cells were incubated with lenalidomide for 48 hours at 37° C. and then collected and washed twice with PBS followed by fixation with 2% paraformaldehyde. Cells were then attached on a slide glass using Cytospin (Thermo Electron, Waltham, Mass.) and permeablilized with cold acetone for 5 minutes. Before cell membrane and nucleus staining, cells were preincubated with 1% bovine serum albumin in PBS for 1 hour at room temperature to reduce nonspecific binding. Cells were incubated with 50 μg/mL concanavalin A (Invitrogen) for 5 minutes at room temperature to stain cell membrane and with 1 μg/mL DRAQ5TM (Biostatus, Leicestershire, United Kingdom) for 5 minutes at room temperature to stain nucleus. Finally, slides were rinsed twice with PBS and covered with glass for confocal microscopy observation. Green fluorescence of fluorescein isothiocyanate-CD20, red fluorescence of concanavalin A, and blue fluorescence of DRAQ5 were analyzed, and merged images were produced using Zeiss 510 META Laser Scanning Confocal Imaging Systems and LSM Image software (Carl Zeiss, Thornwood, N.Y.).

Statistical Analyses

All analyses were performed by the Center for Biostatistics, The Ohio State University. Mixed effects models were fitted to the cytotoxicity data. Random effects associated with the interactions were always included in the models to ensure that error was not underestimated. SPSS software, version 13.0 (SPSS, Chicago, Ill.) was used for the statistical analysis. Nonparametric paired Wilcoxon signed ranked tests were performed to examine both the proportion and mean fluorescence intensity change of cells expressing specific antigens after treatment with lenalidomide or media for 48 hours. A paired t test was used to evaluate whether there was a change in CD20 expression level between cells treated with lenalidomide and untreated control.

Results

Lenalidomide Causes Down-regulation of the CD20 Antigen in Primary CLL Cells

The inventors herein assessed the ability of lenalidomide to promote apoptosis in CLL subject cells. CLL cells incubated with 0.5 μM lenalidomide for 48 or 72 hours exhibited no apoptosis in vitro (data not shown) in agreement with previous reports.²⁸

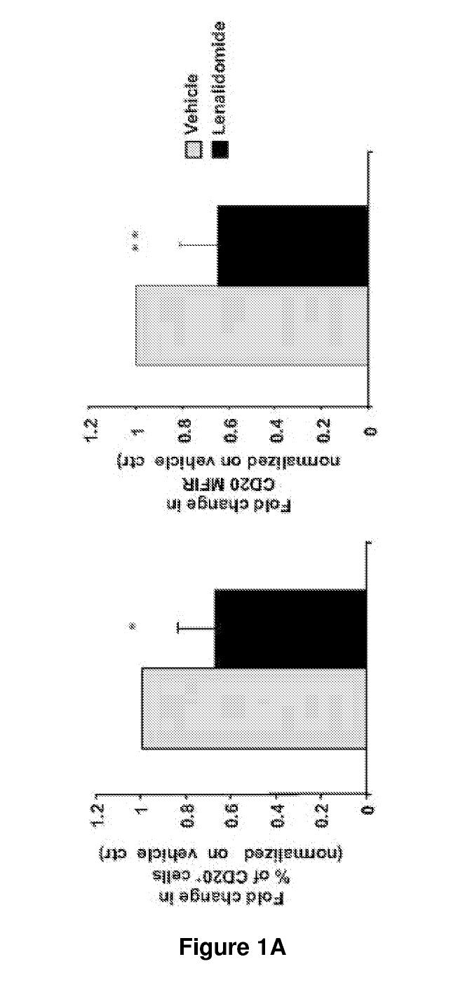

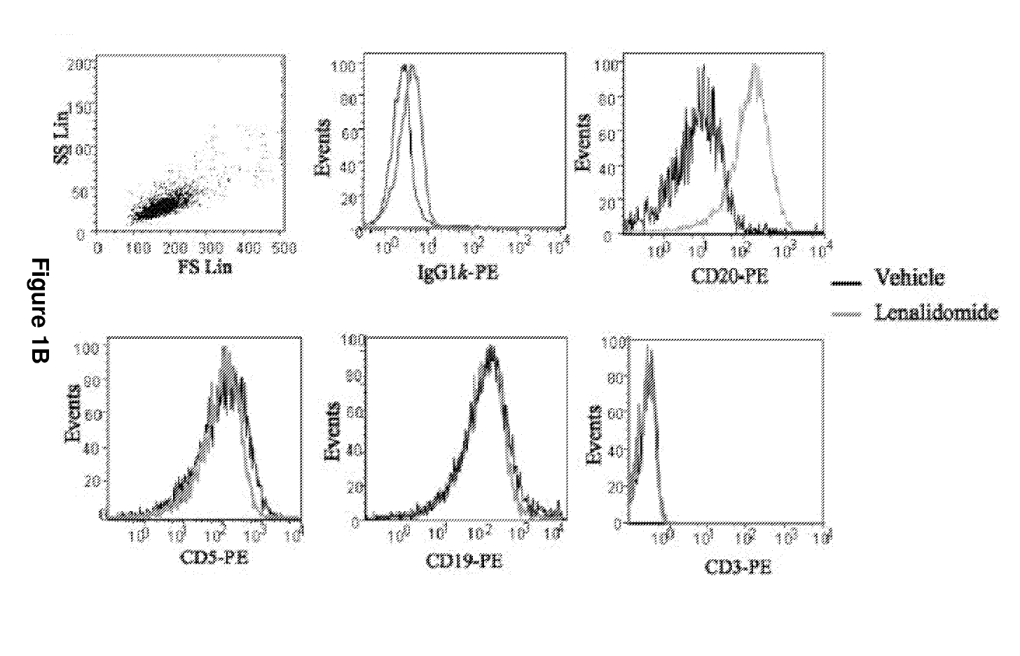

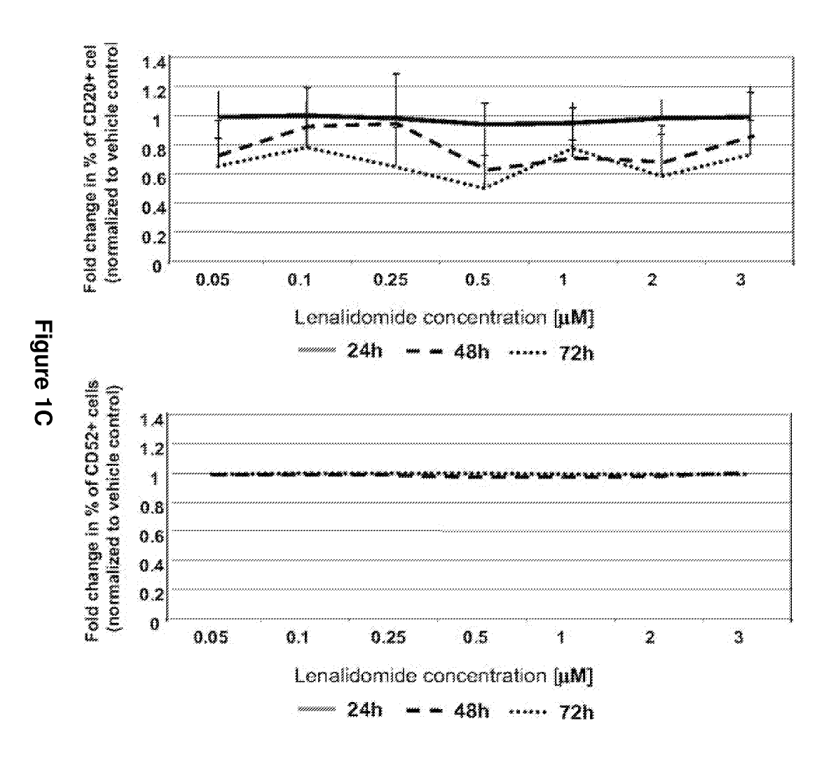

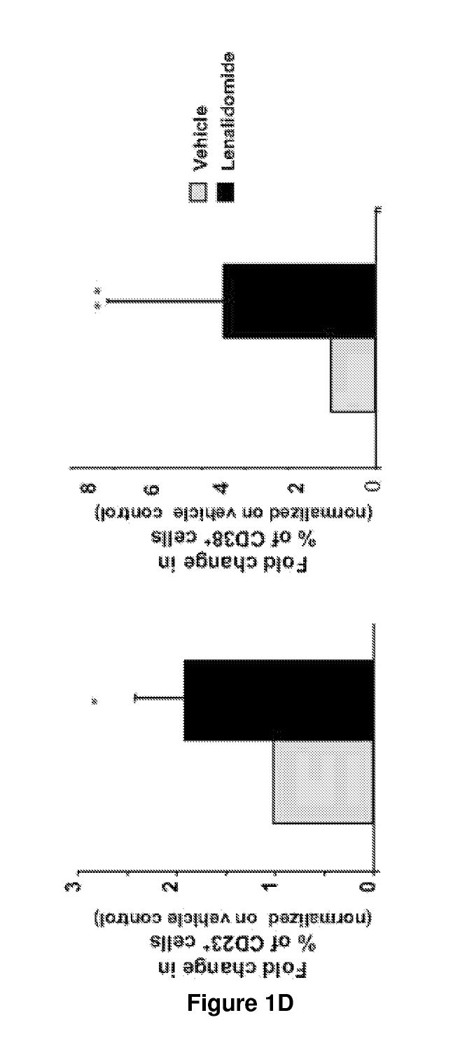

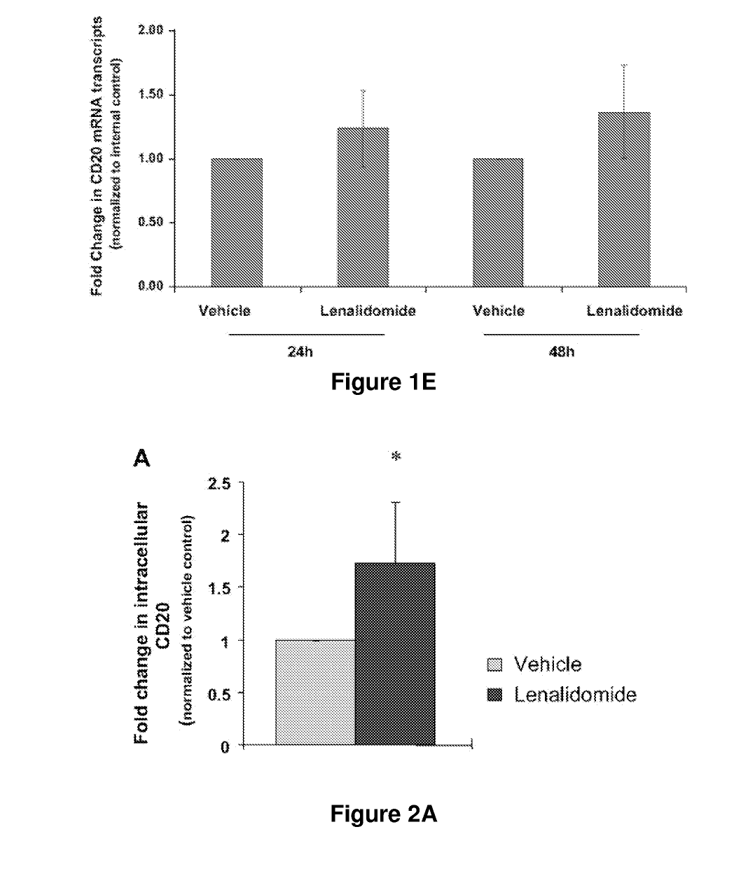

However, as shown in FIG. 1A, at least 40% reductions in the proportion (left panel, P<0.001) and relative mean fluorescence intensity (right panel, P=0.001) of CD20-expressing cells were observed in samples treated with lenalidomide compared with vehicle-treated controls (n=18).

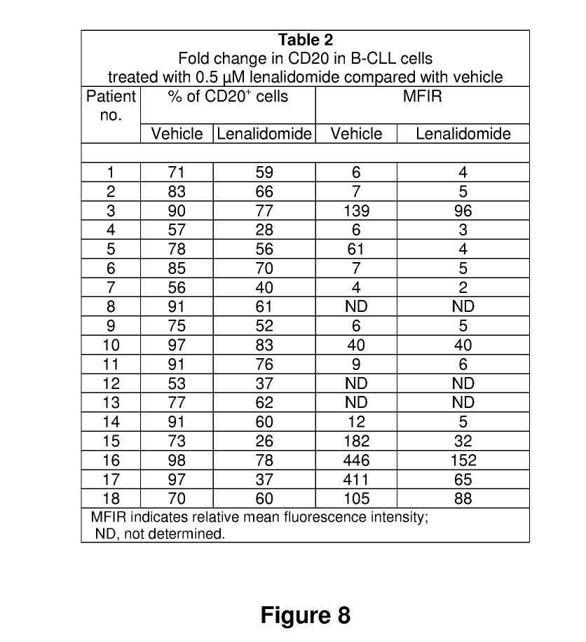

FIG. 8—Table 2 reports CD20 relative mean fluorescence intensity and percentage values for all the samples analyzed. No differences were observed between vehicle (PBS)-treated and untreated cells (data not shown); therefore, vehicle treatment was chosen as experimental control for all the in vitro studies. Lenalidomide treatment induced also increased levels of CD23 and CD38 activation markers on CLL cells (P=0.001 and P<0.05, respectively, FIG. 1D); whereas, no changes in CD19, CD5, and CD52 (data not shown) expression was observed. A representative case is shown in FIG. 1B.

The effect of a wider dose range of lenalidomide (0.05-3 μM) on the CLL cells was tested. The levels of CD20 and CD52 antigens were evaluated at 24, 48, and 72 hours (FIG. 1C) for each dose. The maximum effect on CD20 down-regulation was achieved at 72 hours with 0.5 μM lenalidomide. Thus, this concentration was chosen to perform all the in vitro studies described herein.

The inventors herein then determined whether the mechanism by which lenalidomide down-regulates CD20 antigen was via decreased transcription. CD20 mRNA expression level was assessed by real-time reverse-transcribed polymerase chain reaction (RT-PCR) in lenalidomide- and vehicle-treated CLL cells at 24 and 48 hours.

FIG. 1E demonstrates that lenalidomide treatment resulted in a very modest increase in CD20 mRNA transcription at 24 and 48 hours, as compared with vehicle control. Previous reports have suggested that, in the setting of B-cell activation, CD20 can be internalized.²⁹

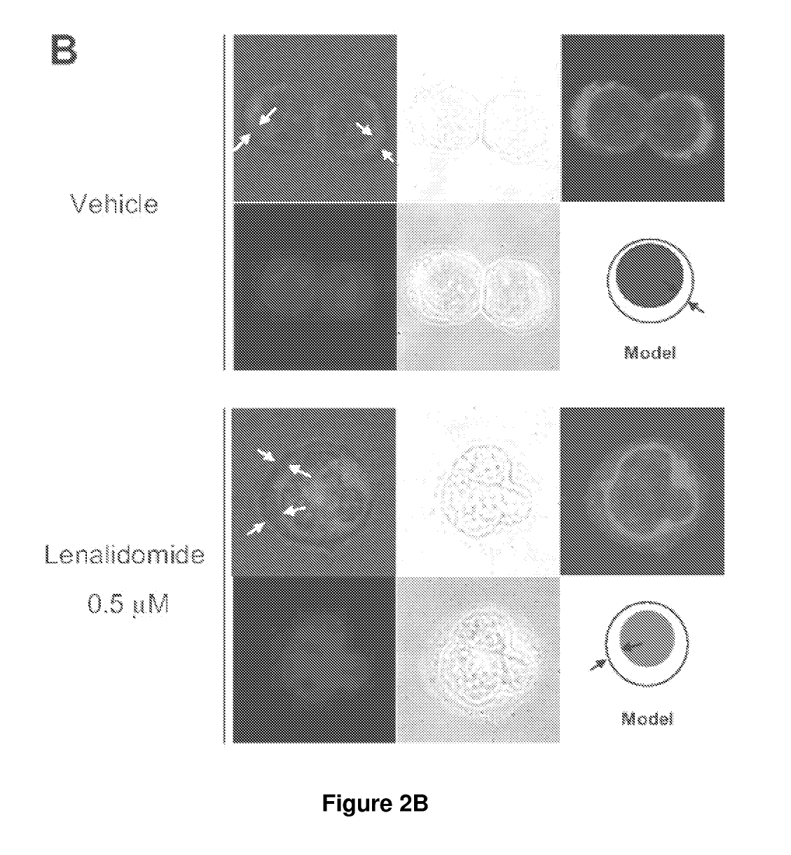

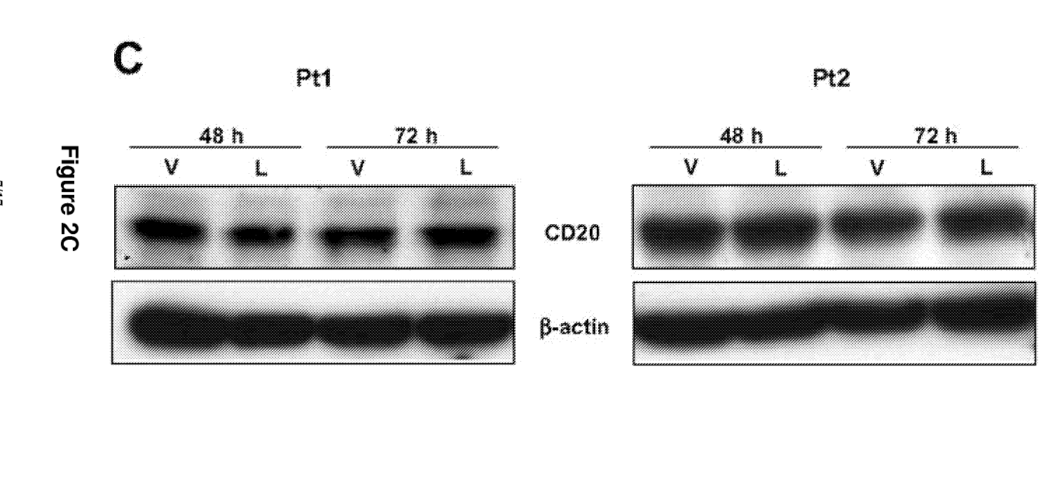

The inventors determined whether the lenalidomide-mediated reduction in CD20 expression (observed by flow cytometry) was the result of CD20 internalization. Reduction of CD20 antigen surface expression was observed in 6 of 8 CLL subject samples after lenalidomide treatment, and this was associated with increased intracellular fluorescence compared with vehicle-treated controls (P=0.006; FIG. 2A).

This was further confirmed by confocal microscopy analysis (FIG. 2B). These results show that CD20 is actively internalized in CLL cells after lenalidomide treatment. An immunoblot analysis of CLL cells treated with lenalidomide for 48 and 72 hours showed no variation in the total level of CD20 protein relative to vehicle control (FIG. 2C).

Lenalidomide-mediated Internalization of CD20 Enhances Intracellular Delivery of ODN in Rituximab Immunoliposomes

While not wishing to be bound by theory, the inventors herein reasoned that, given the internalization of the CD20 antigen after lenalidomide treatment, this may enhance CD20-liposome-mediated intracellular drug delivery.

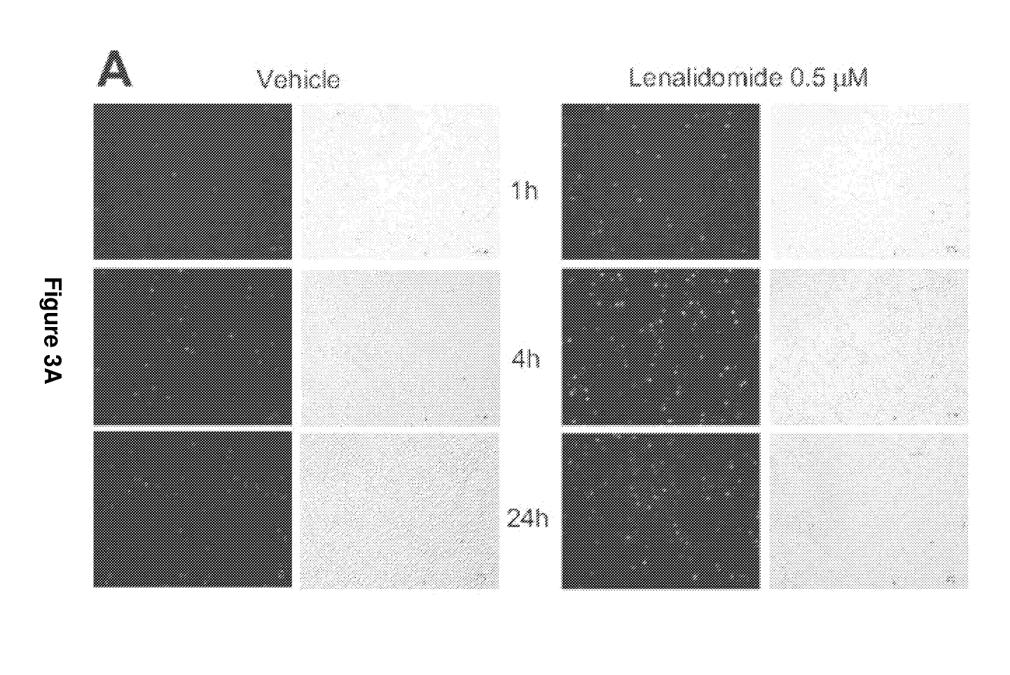

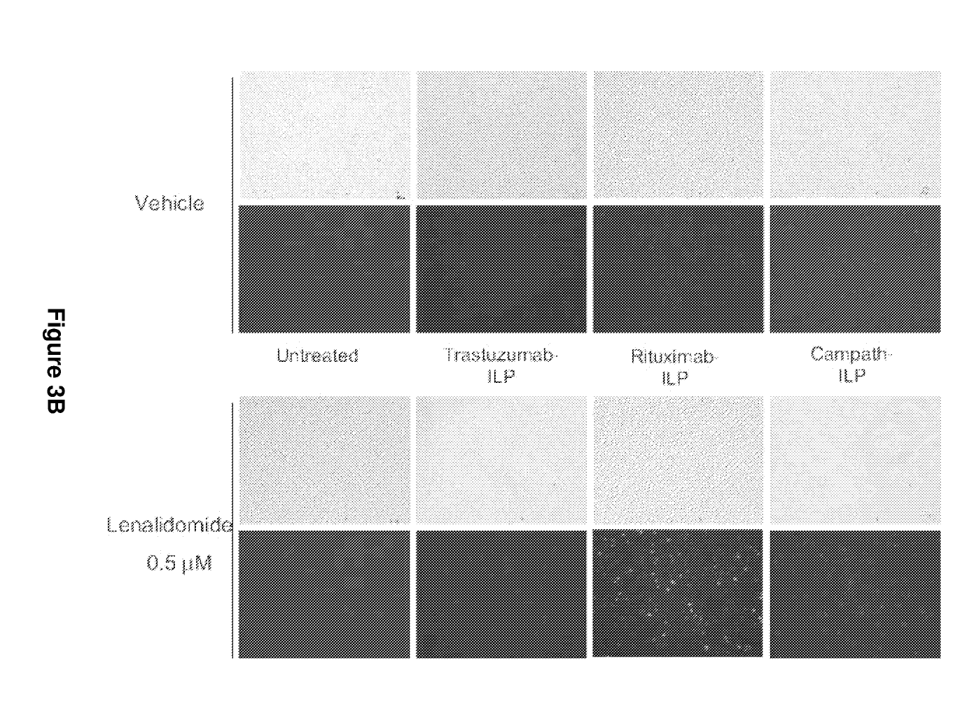

The inventors herein used a FAM-ODN formulated into rituximab-coated immunoliposomes. CLL cells were incubated at 10⁶ cells/mL for 1 hour at 37° C. with rituximab-coated immunoliposome-FAM-ODN (C_{ODN}=1.0 μM) and washed twice in PBS. Cells were kept in culture in RPMI 10% FBS for an additional 1, 4, or 24 hours in the presence of either 0.5 μM lenalidomide or vehicle control. Extracellular bound rituximab immunoliposomes were then washed. Cells were visualized on a Nikon fluorescence microscope (magnification, ×200). Lenalidomide treatment enhanced internalization of fluorochrome-labeled ODN in CLL samples as early as 1 hour after treatment with a maximum effect seen at 24 hours (N=3; FIG. 3A).

In contrast, no enhancement of internalization of fluorochrome-labeled ODN was noted in vehicle-treated cells. This phenomenon requires binding of the CD20 antigen as demonstrated by the lack of fluorochrome-labeled ODN delivery using trastuzumab-coated immunoliposome (FIG. 3B).

Furthermore, no differences were observed in the internalization of fluorochrome-labeled ODN with alemtuzumab immunoliposome between lenalidomide- and vehicle-treated cells (in agreement with the observation that lenalidomide does not modulate the levels of the CD52 antigen).

These examples show that lenalidomide causes antigen-specific internalization of CD20 on CLL cells that leads to enhanced delivery of a fluorochrome-labeled ODN.

Lenalidomide Treatment Antagonizes Rituximab-mediated Apoptosis in CLL Cells

After the inventors examined the modulation of CD20 antigen on CLL cells by lenalidomide, the inventors next assessed whether this treatment adversely influenced cell death promoted by rituximab.

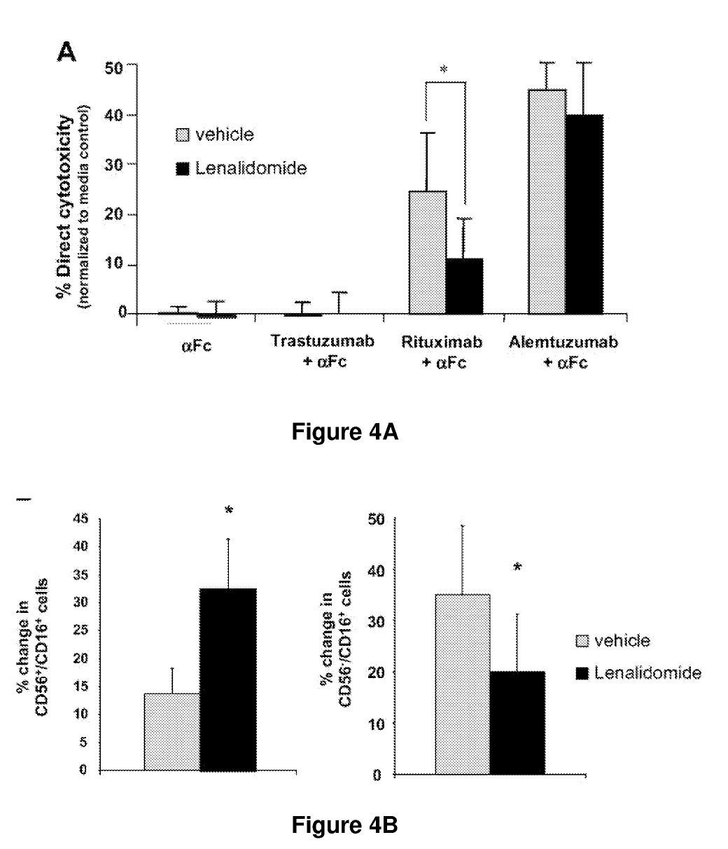

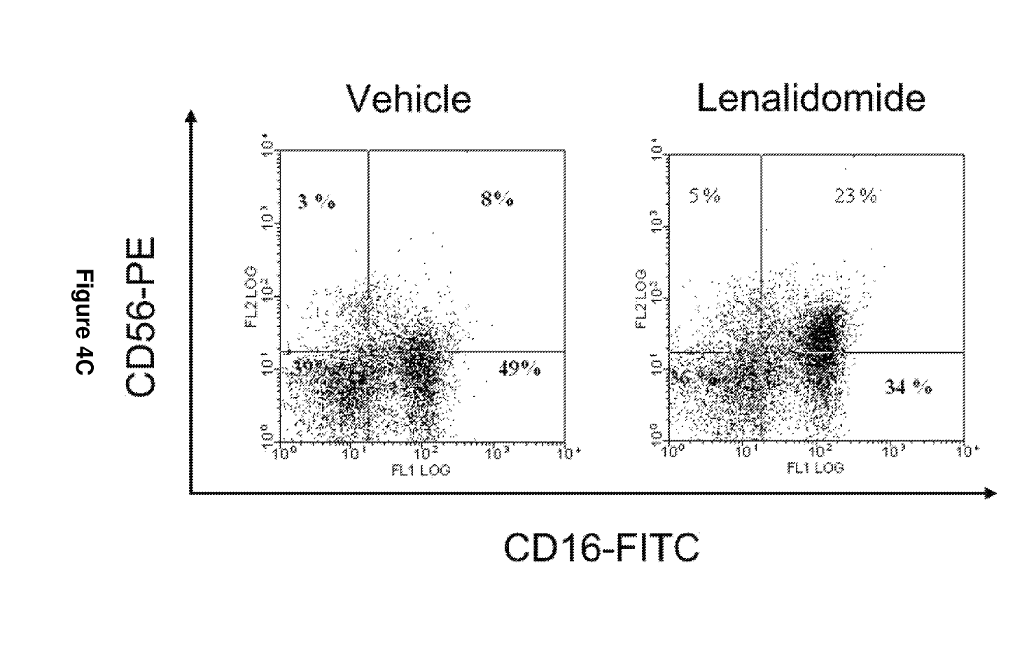

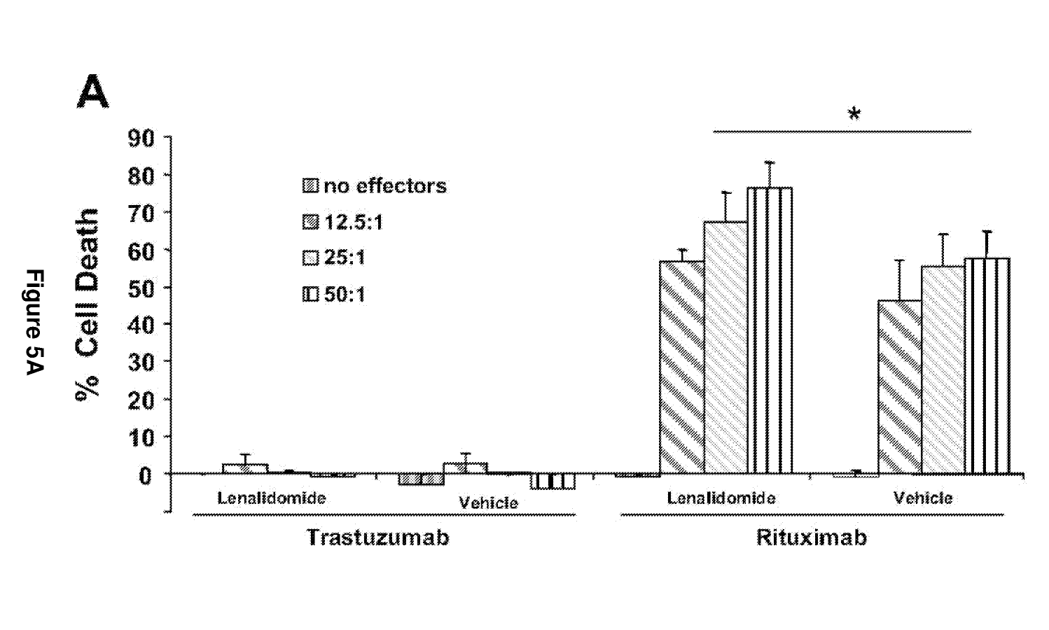

Lenalidomide treatment at 48 hours significantly decreased antibody-mediated apoptosis promoted by rituximab in the presence of cross-linking (25% ±11% cell death compared with 11% ±8% in vehicle- and lenalidomide-treated CLL cells, respectively; P<0.017, N=10; FIG. 4A). No significant change in alemtuzumab-mediated apoptosis was observed under these same conditions.

Lenalidomide Treatment Induces CD56 Expression in CD16⁺CD56⁻ Cells and Enhances NK Cell-mediated ADCC

Previous studies have demonstrated that lenalidomide treatment of normal NK cells enhances CD16 expression and ADCC-mediated by the anti-CD40 antibody SGN40.³⁰

The inventors herein have now surprisingly found herein that treatment of purified NK cells with lenalidomide for 72 hours resulted in an increase in the percentage of CD16⁺/CD56⁺ cells (31% ±14% compared with 18% ±10% in lenalidomide- and vehicle-treated NK cells, respectively; N=12, P<0.01; FIG. 4B left panel).

The increase in CD56⁺ cells was associated with a decrease in CDC16⁺/CD56⁻ cells (20% ±11% compared with 35% ±13% in lenalidomide- and vehicle-treated NK cells, respectively; N=12, P<0.005). This shows the activation of competent CD16⁺/CD56⁺ NK cells by lenalidomide (FIG. 4B right panel).

A representative result for CD56 and CD16 surface expression of lenalidomide-treated NK cells is shown in FIG. 4C.

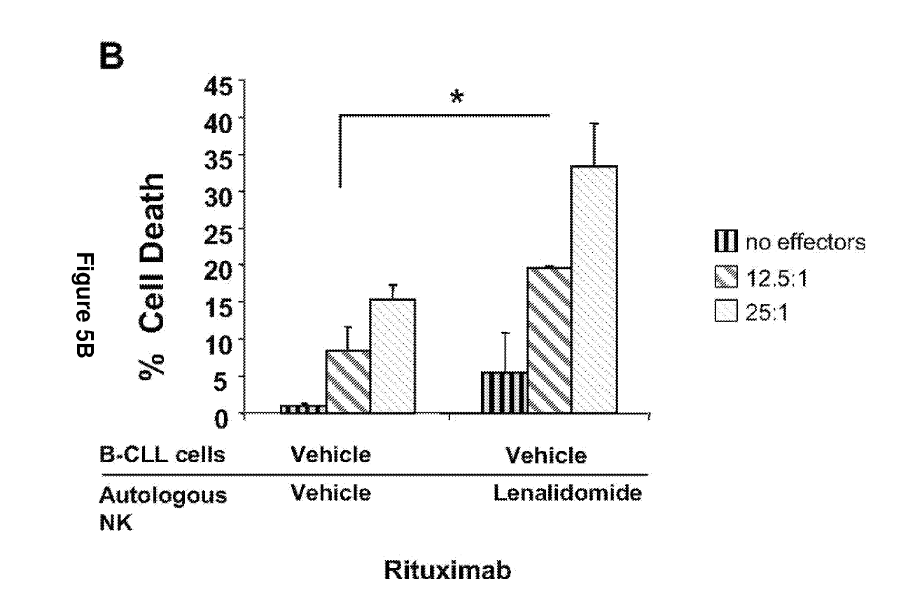

FIG. 5A demonstrates that lenalidomide-treated NK cells from a healthy donor resulted in an overall enhancement of rituximab-mediated ADCC against target CLL subject cells (N=4, P=0.009). Similar results were observed with NK cells derived from subjects with CLL (N=2, P=0.02; FIG. 5B).

Lenalidomide Treatment of CLL Target Cells Antagonizes Rituximab-mediated ADCC

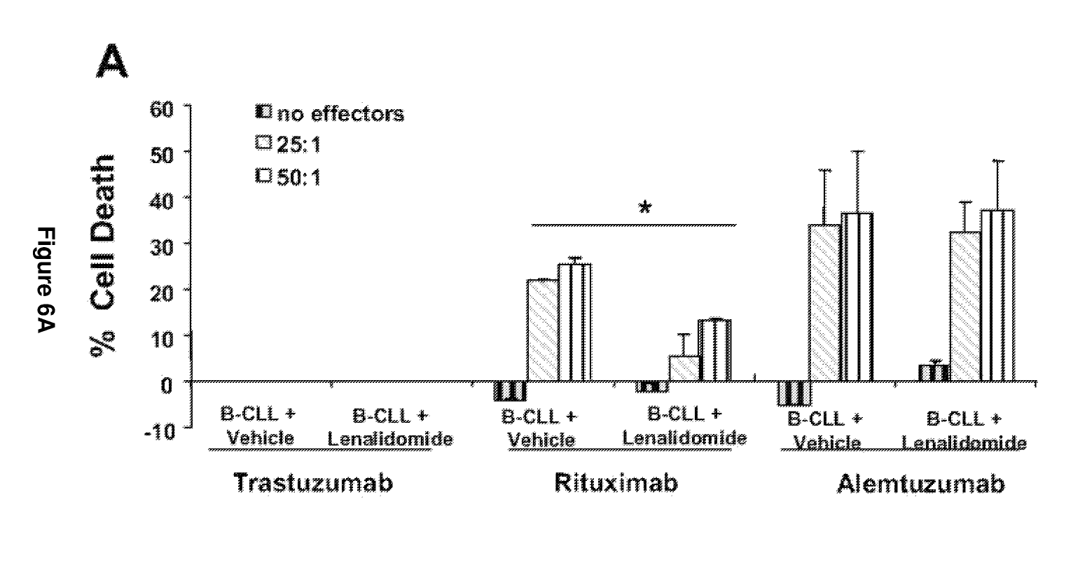

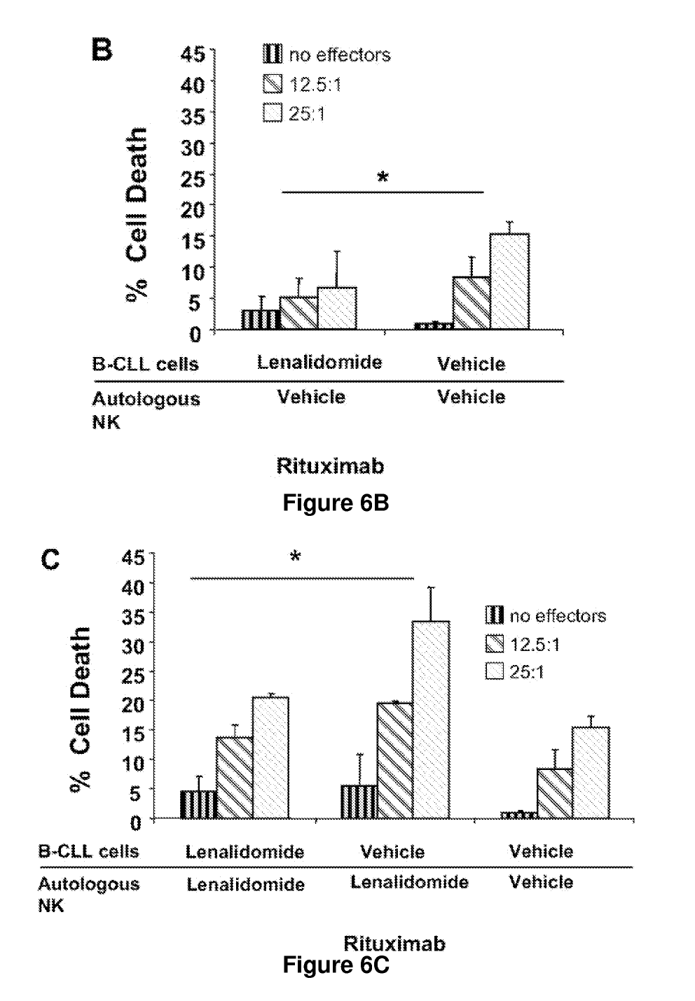

Since the inventors herein have now discovered that lenalidomide induced the down-regulation of CD20 in CLL cells while enhancing NK-mediated ADCC function, the inventors tested the effect of lenalidomide treatment of B cells on CD20-mediated ADCC using rituximab. Pretreatment of CLL B cells at concentrations of lenalidomide previously shown to down-regulate CD20 resulted in a decrease in rituximab-mediated ADCC by either allogeneic (N=5, P<0.05; FIG. 6A) or autologous (N=2, P<0.05; FIG. 6B) NK cells.

As both target CLL cells and NK cells would be subject to lenalidomide effects in vivo, the inventors tested the effect of cotreatment of target CLL B cells and NK cells on rituximab-mediated ADCC function. Peripheral blood mononuclear cells from CLL subjects were incubated for 72 hours with 0.5 μM lenalidomide or vehicle control. After this, B- and NK-cell fractions were purified by magnetic activated cell sorting. Compared with NK-only controls, the cotreatment of autologous NK cells and primary CLL cells with lenalidomide resulted in diminished rituximab-mediated ADCC (N=2, P=0.02; FIG. 6C). In contrast, no significant change in ADCC was observed between lenalidomide versus untreated cells using alemtuzumab (data not shown).

Discussion

The inventors herein have now surprisingly shown that the innate immune-activating agent lenalidomide promotes different biologic effects against subject-derived CLL cells compared with NHL cell lines. Specifically, lenalidomide at concentrations attainable in vivo causes down-regulation of CD20 antigen on the surface of CLL cells.

This CD20 surface antigen down-modulation is not generalized to other surface antigens; i.e., the levels of other B-cell antigens, such as CD52 and CD19, do not change. The mechanism of this lenalidomide-mediated down-regulation of surface CD20 does not involve decreased transcription, but is now believed by the inventors herein to represent internalization of this molecule.

The inventors herein have now surprisingly shown that, via this internalization, the delivery of oligonucleotide to CLL cells via CD20 immunoliposomes is enhanced.

The inventors herein have now surprisingly shown that the simultaneous lenalidomide treatment significantly antagonizes rituximab-mediated apoptosis and ADCC.

In another aspect, there is provided herein a method for treatment of a subject includes the non-simultaneous lenalidomide treatment with rituximab.

The inventors have now also shown that this effect was not generalized, as the effects of alemtuzumab were not altered by lenalidomide treatment. The inventors herein have now demonstrated the complexity of introducing new biologic agents such as lenalidomide with other established agents such as rituximab for CLL.

It is to be noted that the lenalidomide used in these examples was not derived from the pharmaceutical company Celgene (Summit, N.J.) that markets it but rather was acquired after several subjects donated their pharmaceutical-grade medication that they had ceased taking. The lenalidomide was extracted from these tablets using the procedures outlined in “Methods” and confirmed to have very high purity by NMR and liquid chromatography/mass spectrometry analysis performed by The Ohio State University Pharmacoanalytical Shared Resource using a highly sensitive assay.²² The NMR spectra contained only lenalidomide resonance peaks and had no indication of contaminating materials. This same assay is used to measure lenalidomide plasma concentrations as part of ongoing clinical trials at The Ohio State University. Repeated experiments showed consistent biologic activity of lenalidomide as suggested by consistent and reproducible activation of CLL cells and also innate immune effector cells. Obtaining lenalidomide in this manner allowed rapid performance of the research described in this report. In addition, it facilitated performance of other studies where the etiology of tumor flare induced by lenalidomide³¹ or reversal of the T-cell defect³² by this same agent in CLL subjects was reported. The careful analytical assessment performed to assure purity of extracted lenalidomide provides assurance that the reagent used is of high purity. The research performed with extracted lenalidomide also used methodology to assure protection of laboratory research workers from undo exposure to this agent as is typically applied for any biohazardous composition. As such, the use extracted reagent (as performed with lenalidomide in these examples) included bioanalytical assessment to ensure purity of the composition.

NK cells from CLL subjects have been reported to be defective relative to those from healthy volunteers.³³⁻³⁶ The inventors herein now show that autologous NK cells from

CLL subjects demonstrate similar behavior as normal NK cells with respect to enhanced ADCC after treatment with lenalidomide.³⁰ As observed with direct apoptosis, ADCC of CLL target cells was also diminished by lenalidomide treatment using both allogeneic and autologous NK cells. It is to be noted that the inventors herein realize that the use of primary CLL cells as targets, as opposed to B-cell lymphoma cell lines, reduces the ability to fully determine whether this antagonism is the result of CD20 down-regulation and diminished FcγRIIIa binding versus an alteration of the CLL cell that makes it more resistant to NK cell-mediated killing. Also, in other systems with IgG1 antibodies, antigen density on the target cell influences the degree of ADCC observed.³⁷

Also, the effect of antibody-dependent cellular phagocytosis by human monocytes was not modulated by lenalidomide (data not shown).

The inventors herein note that previous studies have demonstrated that activation of CLL cells results in decreased expression of cell surface CD20 expression.³⁸ In prior studies examining the influence of CD40 antigen activation on normal B cells, CD20 surface antigen was observed to decrease. This was not accompanied by changes in transcription but was blocked by protein kinase C inhibitors.

The inventors herein now demonstrate that lenalidomide promotes internalization of CD20 in CLL cells. While not wishing to be bound by theory, the inventors herein now believe that this is through a similar activating mechanism. Indeed, preliminary studies by others³⁹ and several of the co-inventors herein demonstrate that lenalidomide treatment of CLL cells increases costimulatory molecules, including CD86, CD40, CD80, and CD95 as is observed with B-cell activation.

The CD20 antigen generally does not internalize significantly in CLL cells, which is one of the advantages of rituximab.

The inventors herein now show that treatment of CLL cells with lenalidomide greatly enhanced CD20 internalization and, hence, the ability to deliver oligonucleotide therapy using CD20 antibody-mediated immune liposomes. Thus, the present invention also contemplates such non-limiting examples of uses of intracellular delivery of oligonucleotide-based therapeutics, including CpG oligonucleotides, siRNA, and microRNA (which are all relevant to CLL therapy). In particular, the inventors here have how surprisingly found that lenalidomide treatment can significantly improve the efficacy of B cell-specific targeted therapy with CD20 immunoliposomes.

Thus, in another aspect, there is provided herein methods of treatment in the clinical use of lenalidomide in the treatment of CLL and other B-cell malignancies. In particular, shown herein are important differences with lenalidomide between primary CLL cells and B-cell tumor lines.

In addition, the inventors also show that concurrent administration of rituximab with lenalidomide in CLL may, in fact, be counterproductive due to reduction in target expression.

In another aspect, there is provided herein a method of treatment of CCL, comprising the alternative sequences of administration, such as administering rituximab first and then treating with lenalidomide can be considered to be a beneficial therapeutic use.

Described herein is a new therapeutic protocol in which the use of lenalidomide (which represents one of the first biologic agents introduced for the therapy of B-cell malignancies that favorably alters the innate immune system) is carefully evaluated based on its potential influence on target antigens.

While the invention has been described with reference to various and preferred embodiments, it should be understood by those skilled in the art that various changes may be made and equivalents may be substituted for elements thereof without departing from the essential scope of the invention. In addition, many modifications may be made to adapt a particular situation or material to the teachings of the invention without departing from the essential scope thereof.

Therefore, it is intended that the invention not be limited to the particular embodiment disclosed herein contemplated for carrying out this invention, but that the invention will include all embodiments falling within the scope of the claims.

### REFERENCES

The publication and other material used herein to illuminate the invention or provide additional details respecting the practice of the invention, are incorporated by reference herein, and for convenience are provided in the following bibliography.

Citation of the any of the documents recited herein is not intended as an admission that any of the foregoing is pertinent prior art. All statements as to the date or representation as to the contents of these documents is based on the information available to the applicant and does not constitute any admission as to the correctness of the dates or contents of these documents.

1. Lin T S, Moran M, Lucas M, et al. Antibody therapy for chronic lymphocytic leukemia: a promising new modality. Hematol Oncol Clin North Am. 2004;18:895-913.

2. Golay J, Manganini M, Facchinetti V, et al. Rituximab-mediated antibody-dependent cellular cytotoxicity against neoplastic B cells is stimulated strongly by interleukin-2. Haematologica. 2003;88:1002-1012.

3. Golay J, Lazzari M, Facchinetti V, et al. CD20 levels determine the in vitro susceptibility to rituximab and complement of B-cell chronic lymphocytic leukemia: further regulation by CD55 and CD59. Blood. 2001;98:3383-3389.

4. Pedersen I M, Buhl A M, Klausen P, Geisler C H, Jurlander J. The chimeric anti-CD20 antibody rituximab induces apoptosis in B-cell chronic lymphocytic leukemia cells through a p38 mitogen activated protein-kinase-dependent mechanism. Blood. 2002;99:1314-1319.

5. Hatjiharissi E, Xu L, Santos D D, et al. Increased natural killer cell expression of CD16, augmented binding and ADCC activity to rituximab among individuals expressing the FcγRIIIa-158 V/V and V/F polymorphism. Blood. 2007;110:2561-2564.

6. Ghielmini M, Rufibach K, Salles G, et al. Single agent rituximab in subjects with follicular or mantle cell lymphoma: clinical and biologic factors that are predictive of response and event-free survival as well as the effect of rituximab on the immune system. A study of the Swiss Group for Clinical Cancer Research (SAKK). Ann Oncol. 2005;16:1675-1682.

7. Cartron G, Dacheux L, Salles G, et al. Therapeutic activity of humanized anti-CD20 monoclonal antibody and polymorphism in IgG Fc receptor FcgammaRIIIa gene. Blood. 2002;99:754-758.

8. Weng W K, Levy R. Two immunoglobulin G fragment C receptor polymorphisms independently predict response to rituximab in subjects with follicular lymphoma. J Clin Oncol. 2003;21:3940-3947.

9. Khan K D, Emmanouilides C, Benson D M Jr, et al. A phase 2 study of rituximab in combination with recombinant interleukin-2 for rituximab-refractory indolent non-Hodgkin's lymphoma. Clin Cancer Res. 2006;12:7046-7053.

10. Mone A P, Cheney C, Banks A L, et al. Alemtuzumab induces caspase-independent cell death in human chronic lymphocytic leukemia cells through a lipid raft-dependent mechanism. Leukemia. 2006;20:272-279.

11. Farag S S, Flinn I W, Modali R, Lehman T A, Young D, Byrd J C. Fc gamma RIIIa and Fc gamma RIIa polymorphisms do not predict response to rituximab in B-cell chronic lymphocytic leukemia. Blood. 2004;103:1472-1474.

12. List A, Kurtin S, Roe D J, et al. Efficacy of lenalidomide in myelodysplastic syndromes. N Engl J Med. 2005;352:549-557.

13. List A, Dewald G, Bennett J, et al. Lenalidomide in the myelodysplastic syndrome with chromosome 5q deletion. N Engl J Med. 2006;355:1456-1465.

14. Richardson P G, Schlossman R L, Weller E, et al. Immunomodulatory drug CC-5013 overcomes drug resistance and is well tolerated in subjects with relapsed multiple myeloma. Blood. 2002;100:3063-3067.

15. Richardson P G, Blood E, Mitsiades C S, et al. A randomized phase 2 study of lenalidomide therapy for subjects with relapsed or relapsed and refractory multiple myeloma. Blood. 2006;108:3458-3464.

16. Rajkumar S V, Hayman S R, Lacy M Q, et al. Combination therapy with lenalidomide plus dexamethasone (Rev/Dex) for newly diagnosed myeloma. Blood. 2005;106:4050-4053.

17. Chanan-Khan A, Miller K C, Musial L, et al. Clinical efficacy of lenalidomide in subjects with relapsed or refractory chronic lymphocytic leukemia: results of a phase II study. J Clin Oncol. 2006;24:5343-5349.

18. Ferrajoli A, Lee B N, Schlette E J, et al. Lenalidomide induces complete and partial remissions in subjects with relapsed and refractory chronic lymphocytic leukemia. Blood. 2008;111:5291-5297.

19. Hernandez-Ilizaliturri F J, Reddy N, Holkova B, Ottman E, Czuczman M S. Immunomodulatory drug CC-5013 or CC-4047 and rituximab enhance antitumor activity in a severe combined immunodeficient mouse lymphoma model. Clin Cancer Res. 2005;11:5984-5992.

20. Reddy N, Hernandez-Ilizaliturri F J, Deeb G, et al. Immunomodulatory drugs stimulate natural killer-cell function, alter cytokine production by dendritic cells, and inhibit angiogenesis enhancing the anti-tumour activity of rituximab in vivo. Br J Haematol. 2008;140:36-45.

21. Cheson B D, Bennett J M, Greyer M, et al. National Cancer Institute-sponsored Working Group guidelines for chronic lymphocytic leukemia: revised guidelines for diagnosis and treatment. Blood. 1996;87:4990-4997.

22. Liu Q, Farley K L, Johnson A J, et al. Development and validation of a highly sensitive liquid chromatography/mass spectrometry method for simultaneous quantification of lenalidomide and flavopiridol in human plasma. Ther Drug Monit. 2008;118:2427-2437.

23. Mone A P, Huang P, Pelicano H, et al. Hu1D10 induces apoptosis concurrent with activation of the AKT survival pathway in human chronic lymphocytic leukemia cells. Blood. 2004;103:1846-1854.

24. Livak K J, Schmittgen T D. Analysis of relative gene expression data using real-time quantitative PCR and the 2(-Delta Delta C(T)) method. Methods. 2001;25:402-408.

25. Chiu S J, Liu S, Perrotti D, Marcucci G, Lee R J. Efficient delivery of a Bcl-2-specific antisense oligodeoxyribonucleotide (G3139) via transferrin receptor-targeted liposomes. J Control Release. 2006;112:199-207.

26. Akerstrom B, Brodin T, Reis K, Bjorck L. Protein G: a powerful tool for binding and detection of monoclonal and polyclonal antibodies. J Immunol. 1985;135:2589-2592.

27. Akerstrom B, Bjorck L. A physicochemical study of protein G, a molecule with unique immunoglobulin G-binding properties. J Biol Chem. 1986;261:10240-10247.

28. Chanan-Khan A, Porter C W. Immunomodulating drugs for chronic lymphocytic leukaemia. Lancet Oncol. 2006;7:480-488.

29. Anolik J, Looney R J, Bottaro A, Sanz I, Young F. Down-regulation of CD20 on B cells upon CD40 activation. Eur J Immunol. 2003;33:2398-2409.

30. Tai Y T, Li X F, Catley L, et al. Immunomodulatory drug lenalidomide (CC-5013, IMiD3) augments anti-CD40 SGN-40-induced cytotoxicity in human multiple myeloma: clinical implications. Cancer Res. 2005;65:11712-11720.

31. Andritsos L, Johnson A J, Lozanski G, et al. Higher doses of lenalidomide are associated with unacceptable toxicity including life threatening tumor flare in subjects with chronic lymphocytic leukemia. J Clin Oncol. 2008;26:2519-2525.

32. Ramsay A G, Johnson A J, Lee A M, et al. Chronic lymphocytic leukemia T cells show impaired immunological synapse formation that can be reversed with an immunomodulating drug. J Clin Invest. 2008;118:2427-2437.

33. Ziegler H W, Kay N E, Zarling J M. Deficiency of natural killer cell activity in subjects with chronic lymphocytic leukemia. Int J Cancer. 1981;27:321-327.

34. Kay N E, Zarling J M Impaired natural killer activity in subjects with chronic lymphocytic leukemia is associated with a deficiency of azurophilic cytoplasmic granules in putative NK cells. Blood. 1984;63:305-309.

35. Katrinakis G, Kyriakou D, Papadaki H, Kalokyri I, Markidou F, Eliopoulos G D. Defective natural killer cell activity in B-cell chronic lymphocytic leukaemia is associated with impaired release of natural killer cytotoxic factor(s) but not of tumour necrosis factor-alpha. Acta Haematol. 1996;96:16-23.

36. Jakobisiak M, Janowska-Wieczorek A, Dobaczewska H, et al. Decreased antibody-dependent cellular cytotoxicity in various types of leukaemia in man. Scand J Haematol. 1981;27:181-185.

37. Velders M P, van Rhijn C M, Oskam E, Fleuren G J, Warnaar S O, Litvinov S V. The impact of antigen density and antibody affinity on antibody-dependent cellular cytotoxicity: relevance for immunotherapy of carcinomas. Br J Cancer. 1998;78:478-483.

38. Valentine M A, Cotner T, Gaur L, Torres R, Clark E A. Expression of the human B-cell surface protein CD20: alteration by phorbol 12-myristate 13-acetate. Proc Natl Acad Sci U S A. 1987;84:8085-8089.

39. Padmanabhan S, Wallace P, Miller K C, et al. First clinical evidence of in vivo natural killer (NK) cell modulation in chronic lymphocytic leukemia (CLL) subjects (pts) treated with lenalidomide (L) [abstract]. Blood. 2006;108: a. Abstract 2109.

40. Durig J, Ebeling P, Grabellus F, et al. A novel nonobese diabetic/severe combined immunodeficient xenograft model for chronic lymphocytic leukemia reflects important clinical characteristics of the disease. Cancer Res. 2007;67:8653-8661.

1. A method of treating a B-cell malignancy in a subject, comprising using a sequencing strategy to avoid antagonism between an innate immune enhancing agent comprising lenalidomide and an anti-CD20 antibody comprising rituximab in a therapeutic regimen for the subject.

2. The method of claim 1, wherein the rituximab and the lenalidomide are administered sequentially.

3. The method of claim 1, wherein the B-cell malignancy comprises chronic lymphocytic leukemia (CLL).

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. A method of inhibiting or reducing a B-cell malignancy comprising: screening subjects to identify those in which expression of CD20 is upregulated; and administering an antibody that binds to and inhibits CD20; and thereafter administering an innate immune enhancing agent that internalizes CD20.

16. The method of claim 15, wherein the anti-CD20 antibody comprises rituximab and the innate immune enhancing agent comprises lenalidomide.

17. A method for increasing sensitivity of a B-cell to a therapeutic agent, comprising contacting the B-cell with a therapeutically effective amount of rituximab, and thereafter contacting the B-cell with a therapeutically effective amount of lenalidomide.

18. The method of claim 17, wherein the B-cell is from a subject suffering from chronic lymphocytic leukemia (CLL).

19. A method for treating a subject for a cancer or pre-malignant condition that is associated with CD20-expressing cells, the method comprising:
administering to the subject a therapeutically or prophylactically effective amount of an anti-CD20 antibody comprising rituximab,
followed by administering to the subject a therapeutically or prophylactically effective amount of an innate immune enhancing agent comprising lenalidomide.

20. The method of claim 19, wherein the cancer or pre-malignant condition is a cancer of B-cell lineage.

21. The method of claim 20, wherein the cancer of B-cell lineage is chronic lymphocytic leukemia (CLL).

22. (canceled)

23. (canceled)

24. (canceled)

25. (canceled)

26. (canceled)

27. (canceled)

28. (canceled)

29. (canceled)

30. (canceled)

31. (canceled)

32. A kit for administering a first and a second pharmaceutical composition to a subject suffering from a B-cell malignancy, the kit comprising:
i) a plurality of separate containers, the contents of at least two containers differing from each other in whole or in part,
wherein at least one of such containers contains an innate immune enhancing agent, with or without additional pharmaceutical carrier or diluent, and at least one different container contains an anti-CD20 antibody, with or without additional pharmaceutical carrier or diluent; and,
ii) instructions for the use of the contents of the containers after an interval of time has passed after administration of the first pharmaceutical composition for the treatment of a subject suffering from a B-cell malignancy;
wherein the treatment:
(i) is the sequential administration to the subject of first an anti-CD20 antibody and then an innate immune enhancing agent after an interval of time has passed; or
(ii) results in the sequential contact of a B-cell included in, derived from or being part of the B-cell malignancy with first an anti-CD20 antibody and then an innate immune enhancing agent after an interval of time has passed,
(iii) is the sequential administration to the subject of first an innate immune enhancing agent and then an anti-CD20 antibody after an interval of time has passed; or
(iv) results in the sequential contact of a B-cell included in, derived from or being part of the B-cell malignancy with first an innate immune enhancing agent and then an anti-CD20 antibody after an interval of time has passed; and
wherein the administration:
i) is the sequential administration to the subject of an anti-CD20 antibody and an innate immune enhancing agent within from 1 day to 10 days of each other; or
ii) results in the sequential contact of a cell included in, derived from or being part of the B-cell malignancy with an anti-CD20 antibody and an innate immune enhancing agent within from 1 day to 10 days of each other.

33. A method to inhibit one or more B-cell malignancies or metastases in a subject, the method comprising the sequential steps of:
(a) administering to the subject an effective amount of an anti-CD20 antibody comprising rituximab;
(b) waiting for an interval of time; and
(c) administering to the subject an effective amount of an innate immune enhancing agent comprising lenalidomide, wherein the B-cell malignancies or metastases decrease more than would be the case for an otherwise identical method that lacks step (b).

34. The method of claim 33, wherein the B-cell malignancies or metastases express a receptor for the innate immune enhancing agent.

35. The method of claim 33, wherein step (b) comprises waiting longer than about five days.

36. The method of claim 33, wherein step (b) comprises waiting longer than about ten days.

37. The method of claim 33, wherein the anti-CD20 antibody and/or the innate immune enhancing agent are administered by introducing into the subject one or more exogenous nucleic acid constructs encoding the anti-CD20 antibody an/or the innate immune enhancing agent, in a manner permitting expression of the anti-CD20 antibody and/or the innate immune enhancing agent.

38. The method of claim 37, wherein the expression of the anti-CD20 antibody and the innate immune enhancing agent are in a time sequenced interval.

39. The method of claim 38, wherein the interval of time between the administration of the anti-CD20 antibody and the administration of the innate immune enhancing agent is one day to two months.

40. A method to diagnose the likely responsiveness of a B-cell malignancy to the sequential administration of an anti-CD20 antibody followed by the administration of an innate immune enhancing agent, the method comprising
analyzing cells from the B-cell malignancy for the presence of a receptor,
wherein the presence of the receptor indicates that the B-cell malignancy will likely respond to the sequential administration of the anti-CD20 antibody followed by the administration of the innate immune enhancing agent.

41. (canceled)

42. (canceled)

43. (canceled)

44. (canceled)

45. The method of claim 33, wherein the interval of time between the administration of the anti-CD20 antibody and the administration of the innate immune enhancing agent is one day to two months.

46. The method of claim 33, wherein the administration:
a) is the sequential administration to the subject of the anti-CD20 antibody and the innate immune enhancing agent within about 10 days, 7 days, 5 days, 3 days, 2 days or 1 day of each other; or
(ii) results in the sequential contact of a B-cell included in, derived from or being part of the B-cell malignancy with the anti-CD20 antibody and the innate immune enhancing agent within about 10 days, 7 days, 5 days, 3 days, 2 days or 1 day of each other.

47. The method of claim 33, wherein the administration:
a) is the sequential administration to the subject of the anti-CD20 antibody and the innate immune enhancing agent within about 48 hours, 24 hours, 12 hours, 8 hours, 6 hours, 4 hours, 2 hours, 1 hour, 30 mins, 15 mins or 5 mins of each other; or
b) results in the sequential contact of a B-cell included in, derived from or being part of the B-cell malignancy with the anti-CD20 antibody and the innate immune enhancing agent within about 48 hours, 24 hours, 12 hours, 8 hours, 6 hours, 4 hours, 2 hours, 1 hour, 30 mins, 15 mins or 5 mins of each other.

48. (canceled)

49. An oral dosage form comprising an effective amount of an anti-CD20 antibody comprising rituximab and an innate immune enhancing agent comprising lenalidomide in a combined form,
wherein the oral dosage form is coated with a time-dependent release coating material that delays release of the lenalidomide until after rituximab has been released.

50. (canceled)
